(19) United States
(12) Patent Application Publication
(10) Pub. No.: US 2011/0313218 A1
(43) Pub. Date: Dec. 22, 2011

(54) Systems, Apparatus and Methods of a Dome Retort

(76) Inventor: Todd C. DANA, Park City, UT (US)

(21) Appl. No.: 13/070,334

(22) Filed: Mar. 23, 2011

Related U.S. Application Data
(60) Provisional application No. 61/316,748, filed on Mar. 23, 2010.

(51) Int. Cl. C07C 1/00; B01J 19/18; C10C 1/20; C10G 1/00

(52) U.S. Cl. 585/240; 208/400; 208/409; 422/164; 422/233; 196/46

(57) ABSTRACT

A system, apparatus and method for hydrocarbon extraction from feedstock material that is or includes organic material, such as oil shale, coal, lignite, tar sands, animal waste and biomass. A retort system including at least one retort vessel may include a monolithic dome structure surrounded by a process isolation barrier, the dome structure being sealingly engaged with the process isolation barrier. The dome structure and the process isolation barrier define a retort chamber, at least a portion of which may comprise a subterranean chamber. A lower end of the dome retort structure provides an exit for collected hydrocarbons and spent feedstock material. Systems may include a plurality of such dome retort structures. A control system may be used for controlling one or more operating parameters of a retorting process performed within such a dome retort structure for extraction and collection of hydrocarbons.

### PRIORITY CLAIM

This application claims the benefit of and priority from U.S. Provisional Patent Application No. 61/316,748 filed on Mar. 23, 2010 that is incorporated in its entirety for all purposes by this reference.

### FIELD

Embodiments of the invention relate generally to extraction of hydrocarbons from organic materials and, more specifically, to extraction of hydrocarbons from organic materials in a substantially continuous process employing a substantially dome retort, employed in the system and associated methods.

### BACKGROUND

Billions of barrels of oil remain locked up in oil shale, coal, lignite, tar sands, animal waste and biomass around the world, yet an economically viable, easily scalable hydrocarbon extraction process has not, to date, been developed. Few, if any, extraction processes are even in commercial use without government subsidies. Throughout the history of unconventional fuel extraction by pyrolysis, many various types of retorting processes have been used, but in general, there are similar genres for these processes. The genres of technologies have generally been categorized as i) above-ground retorts, ii) in-situ processes, iii) modified in-situ processes, and iv) above-ground capsulation processes. Each genre in the prior art exhibits specific benefits, but also associated problems which preclude successful unsubsidized commercial implementation.

Above-Ground Retorts

Above-ground retorts in the form of fabricated vessels may be of many sizes shapes and designs, offering various attributes in terms of throughput rate, heat recovery, heat source type and horizontal or vertical engineering. Technologies for above-ground retorting include, but are not limited to, plants and facility designs such as those of Petrosix, Fushun, Parahoe, Kiviter and the AlbertaTaciuk Process (ATP), among others. In general, all of these processes are examples of above-ground and fabricated steel retorts which move heated rock through them.

Success of conventional, above-ground retorting has been severely limited due to economic factors. Among the many economic considerations precluding failed commercialization include the cost of fabrication, requiring large volumes of steel, complex forming and welding, compounded by the need to construct ever-larger retorts simply to handle a sufficiently large feedstock ore of hydro carbonaceous material (such as, for example, oil shale) volume to achieve hydrocarbon production on a large-enough scale to justify transportation (pipeline) infrastructure leading to a refinery, or a refinery on site. The perception is that, for retort-based hydrocarbon production on a commercial scale, one must have rapid feedstock ore throughput in order to achieve volume economics; however, any increased feedstock ore throughput must, conventionally also require an increase in heat rate and, therefore, temperature of the overall retort. Yet, by going to a higher retorting temperature, the quality of the produced hydrocarbons decreases and the higher temperature creates a substantially higher volume of emissions than is desirable, or even permissible under ever-mare-restrictive government regulations. Further contributing to the problems of this technology is the requirement for economic viability that the increased heat rate and higher temperature associated with a faster feedstock ore throughput compels the recovery of more energy from the feedstock ore prior to discharge. These energy input and recovery problems associated with conventional retort-based technology are directly related to its poor economic performance.

Another common denominator leading to failure for above-ground retorts is the limitation of retort size. Economically and practically speaking, an above-ground steel retort cannot be built large enough, due to both difficulties in fabrication of a large enough retort vessel as well as required support structure to enable a sufficiently long residence time for feedstock ore at a relatively low temperature to provide adequate throughput. Thus, the limited sizes of above-ground retorts requires a short heating residence time within but, as noted above, the faster, higher heat rate then yields a lower quality oil and greater heat recovery challenge so as not to destroy economics of the process by losing energy efficiency.

Further to the challenge is the economy and efficiency of scale in production and processing. For example, several of the largest oil shale retorts in the world including the Stuart Shale Project, the Parahoe, the ATP, and the PetroSix, each produce less than 5,000 barrels (bbl) per day. Some of these have never run at steady state or anywhere even near this cited volume. Relative to large oil wells and relative to the capital for these wells, oil shale and coal retorting becomes unattractive economically given the low volume output juxtaposed by the high capital cost. Further, most liquids from pyrolysis require the additional processing step of hydrotreating to remove arsenic, nitrogen and other undesirable chemical attributes in oil. But because of the economy of scale issue also impacting the capital cost and operating cost of hydrotreating plants necessary to remove nitrogen, add hydrogen and remove arsenic, these facilities also depend on an oil feedstock rate in quantities of at least 20,000 bbls per day to justify the construction of these multi-hundred million dollar facilities. Accordingly, great volume to justify costs in the upstream production (pyrolysis) and the downstream processing (hydrotreating) are needed and each problem depends on the upstream retorting volumes of a given extraction process.

In-Situ Processes

Difficulties relative to limited retort volume from above-ground retort feedstock ore processing gave rise to the concept and development of leaving such hydrocarbonaceous material in place and heating it in formation, such processes being known as “in-sin. processes” and “modified in-situ processes.” The concept of in-situ processes is based on the assumption that by forgoing the mining and handling of feedstock are in favor of drilling through the formation comprising the hydrocarbonaceous material, you can reduce costs by simply introducing heat into the formation through the resulting bore holes to extract hydrocarbon liquids. The logic seems simple and, therefore, sounds like a good idea on paper. Thus, there have emerged many conceptual approaches to introduce heat below ground by drilling a well pattern in the ground and, in some cases, using so-called “intelligent” geometric spacing in an attempt to efficiently add heat or remove gas and liquids.

In-situ processes, while thermally and economically promising in theory, suffer in practice from an undeniable, industry-blocking problem in the form their inability to effectively protect subterranean hydrology proximate the production area following in-situ heating. It is becoming more appreciated with the passage of time and increase in demand due to residential, agricultural, commercial and industrial development that the one natural resource which is more valuable than crude oil is fresh ground water. For example, in oil shale-rich regions around the world—particularly in the Western United States as well as in the deserts of Australia, Jordan and Morocco—fresh water is in limited supply. In some cases, such as in Colorado's Piceance Basin, the oil shale formation is also in direct contact, both above and below, with the fresh water snow pack runoff from the Rocky Mountains.

In recent years several technologies have made progress relating to in-situ recovery, but none have come up with a 100% effective solution for also protecting ground water following in-situ extraction processes. Even with the advent of Royal Dutch Shell's so-called “freeze wall” technology to solidify moisture in-situ surrounding the process area to protect ground water before and during operation of Shell's in-situ process, Shell has not and cannot provide assurance that ground water contamination will not occur after the freeze wall is allowed to thaw. Over time, ground water returns to the formation containing the post-processed materials and then interacts with the formerly heated zones which still contain remaining volatile organic compounds which will then proceed to migrate and eventually contaminate rivers and streams in the area. Confidence related to hydrology protection is therefore needed long after heating of a formation by an in-situ technology. This environmental confidence will only come with the engineered isolation of spent hydrocarbons and ground water, which in-situ processes have been unable to provide.

Another aspect of concern related to in-situ processes is lack of predictability of the overall recovery rate of hydrocarbons from the oil shale or other hydrocarbonaceous material, such as coal, originally in place within the formation. Because in-situ technologies depend on heat introduction methods which hopefully coax hydrocarbons to emerge from production wells, and because subterranean formations are complicated geological structures, there can be no true certainty as to overall recovery rate from an in-situ treated formation. In the case of governments and other entities which lease mineral rights to oil shale or coal producers using such technologies, because royalties paid them are directly related to the overall recovery rate (in terms of volume recovered) of the hydrocarbons in place, recovery in terms of percentage yield of hydrocarbons in place is important.

Modified In-Situ Processes

There are many so-called modified in-situ processes employing blasting and even vertical columns in the ground; however, none of these approaches utilize a permeability control infrastructure to collect hydrocarbons or to segregate the rubble zones from the adjacent formation. In other words, a selected portion or a formation containing organic materials is drilled and blasted to create a “rubbleized” area, which may comprise a vertical rubble column. In-situ application of heat to, and extraction and collection of hydrocarbons from, the rubbleized material is then effected as described above with respect to traditional in-situ processes.

Both in-situ and modified in-situ hydrocarbon extraction processes may be characterized as “batch” processes, in that organic material containing extractable hydrocarbons is processed in place, i.e., at its site of origin. Therefore, all of the associated infrastructure required for heating the organic material and extracting and collecting hydrocarbons therefrom must be built on site, or transported to the site, and is either left on-site (as in the case of underground components) or, if not worn out during the extraction and collection process, transported to another site for re-use.

In Capsule Technology

The present inventor is also a named inventor on United States and other patent applications relating to a batch-type hydrocarbon extraction process, which may be characterized herein for convenience as the “in capsule” extraction process. The in capsule extraction process generally relates to the batch extraction of liquid hydrocarbons from hydrocarbonaceous material in the form of a feedstock ore body contained in an earthen impoundment. Relevant to this process are the aspects of heating the impounded hydrocarbonaceous material in place while it is substantially stationary.

Stationary extraction of hydrocarbons is problematic for several reasons. First, the aspect of the feedstock ore remaining substantially stationary, (allowing for only ore movement in the form of vertical subsidence during heating), entails a single use, batch impoundment which is processed until the yield of liquid and volatile hydrocarbons decreases to a point where cost/benefit of energy input to hydrocarbon yield dictates termination of the operation. These impoundments may be envisioned as an array or pattern of very large (in terms of length and width), one use, spread out pads of feedstock ore just below the earth's surface, similar to ore pads employed in a heap leaching process in mining. The width of each such ore pad requires a superimposed vapor barrier to contain hydrocarbon volatiles released during the heating of the feedstock ore to be formed directly on top of, and supported by, the ore body being heated as no structural steel or other separate vapor barrier support span is economically feasible. Thus, the only feasible option of resting the vapor barrier on top of the feedstock ore subjects the vapor barrier to subsidence of the ore as liquid and volatile hydrocarbons are removed.

As subsidence occurs, cracking of the vapor barrier resting on top of the heap also occurs. Further to the problem is that integrity of a clay impoundment barrier such as is designed to prevent release of the hydrocarbon volatiles (i.e., as a vapor barrier), is dependent on retained moisture which is driven off by the process heat. So, as heating occurs over time, not only does subsidence of the feedstock ore increase, but at the same time the clay impoundment dries, until I the lack of underlying support of the clay impoundment in combination with its drying and associated loss of both flexibility and impermeability to hydrocarbon volatiles results in cracking as well as increased porosity. While a polymeric liner may be employed in combination with a clay impoundment vapor barrier in an attempt to stop vapor leakage through cracks in the clay caused by subsidence, the high temperature of gases escaping through the cracks in the clay will come in contact with any such liner and at the high process temperatures employed will likely melt such liner, compromising its integrity. This major problem of vapor barrier compromise as a result of subsidence is highly detrimental to the economics of hydrocarbon recovery, as well as protection of the ambient environment. In such cases, given the vapor production of pyrolysis which is known, a significant percentage of the potentially recoverable hydrocarbons may be lost as escaped volatiles which, in turn, contaminate the atmosphere.

The problem of subsidence of the feedstock ore body also gives rise to other problems associated with operation of the in capsule extraction process. Subsidence may exhibit such a great problem over time that horizontal pipes used to heat the ore body must be protected by significant preplanning to adjust for the sinking of the pipes during heating. In addition, heater pipe penetration joints may be required to anticipate and attempt to mitigate the subsidence issue as a cause of heater pipe collapse and bending under the force of a subsiding ore body above them. It has been proposed to employ corrugated metal pipe as a means to provide heater pipe flexure in tandem with the collapse of the subsiding ore body so as avoid heating pipe breakage. However, none of the foregoing techniques can be used to address heat-induced subsidence, sinking, cracking and integrity compromise or a vapor barrier supported by the impounded feedstock ore body.

The cost to create permeability control infrastructures for each impounded feedstock ore body is another problem from which the in capsule extraction process suffers. Because the in capsule extraction process is applied to an ore body impoundment, there is no “throughput” of the hydrocarbonaceous materials whatsoever, but instead as a batch process requires a new containment barrier for every single batch processed. With substantial preparation and earth work related to clay impoundments or other control liners necessary before hydrocarbons can be extracted from each impounded ore body, the cost of creating an entirely new barrier becomes prohibitive. The in capsule extraction process also entails a heat up period that is costly in terms of energy input and time waiting for heat up to produce a high enough temperature in the ore body for hydrocarbon recovery to commence.

Therefore, because of the problem of barrier cracking as a result of subsidence, the problem of cost associated with continuous barrier and impoundment construction, and because of the heat up requirement of time and energy for each batch, a better, new invention for controlling vapor without risk of barrier cracking and without high cost of barrier construction is needed.

While it should be readily apparent, a disadvantage of any batch-type hydrocarbon extraction process, be it in-situ, modified in-situ or in capsule, is the batch production of the extracted liquid hydrocarbons. When such processes result in production after a period of heating, the large volume of the extracted liquid hydrocarbons produced over a relatively short period of time requires either immediate access to a pipeline for transportation to a refinery or a large storage tank volume, in either case driving up the cost of such an installation.

### SUMMARY

The present invention, in various embodiments, may provide straightforward, robust solutions to problems associated with conventional hydrocarbon extraction processes applied to hydrocarbonaceous materials (which may also be characterized as organic materials) such as, by way of example and not limitation, feedstock materials (such term being used to encompass organic materials generally, and not limited to mineral or other rock-based ore materials) in the form of oil shale, coal, lignite, tar sands, animal waste and biomass. Among the advantages that may be offered by implementation of aspects of the present invention are enhanced feedstock material throughput, improved recovery of hydrocarbon volatiles as well as enhanced environment protection provided by a high-integrity process isolation barrier including a surface monolithic structure supported independently of in-process organic material, lower capital cost achieved through reuse of process and control infrastructure, and better integrity assurance of the final lining of spent (processed) material tailings due little or no subsidence and associated cracking of the liner. Additional advantages may include time and cost savings through elimination of repetitive barrier construction associated with batch processing, and the requirement of protracted heat up from a cold start for each batch.

Embodiments of the present invention may provide enhanced assurance of volatile hydrocarbon collection from a transportable mass of feedstock material movable through a geologically surface supported dome infrastructure, which may comprise at least a portion of a retort that is not affected by reduction of feedstock material volume during a heating process employed in hydrocarbon extraction. In some embodiments of the invention, heating may be conducted within a descending process and control infrastructure that is enveloped by at least a portion of a monolithic dome structure, which may be supported by underlying stem walls, footings or basement walls encircling a floor where organic material is piled and retorted. The extraction process may employ a process and control infrastructure in the form of a fabricated pass-through retort system disposed within the dome retort, surrounded and capped by a process isolation barrier. This approach may enable maintenance of a substantially continuous process temperature for ongoing hydrocarbon extraction of feedstock material passing through the dome retort system without a new heat up period after process temperature has been reached subsequent to system startup. Processed feedstock material may be cooled beneath or adjacent the dome retort system after it has exited through the floor of the dome retort by, for example, auger assisted removal. Embodiments of the invention may include reclaimers circling and spinning above the floor of the dome retort, which may pull and/or push the heated and retorted materials to a vapor scaled discharge for such spent material. The spent material may also be characterized as tailings, and may descend through the dome retort into a separate chamber or quenching zone beneath or adjacent the dome retort, which may be used to cool the heated material prior to final conveyance to a clay or other environmentally lined impoundment area where the relatively cooler and now reduced-volume spent material may not compromise the integrity of a previously placed and compacted clay liner, or clay or other barrier cap placed thereover for containment and site remediation.

Embodiments of the invention may employ substantially continuous volume heating of hydrocarbonaceous materials and isolate the heated volume and extraction process from the ambient environment surrounding the dome retort process site, including ambient atmosphere and ground water or surface water, and, likewise, isolate the process site from encroachment by the ambient environment. Among other things, embodiments of the invention may reduce operating costs of hydrocarbon extraction from feedstock material due to the use of large domes already being used in bulk storage systems at ports for dry or wet storage. Embodiments of the invention may go beyond bulk storage, and convert these scalable facilities for use in scalable pyrolysis processing while moving and heating the material within them. The process may reduce or avoid air and groundwater contamination throughout the entire processing and post-processing handling of feedstock material, limit surface area disturbance at the processing site, reduce material handling costs, separate fine particulates from the produced synthetic oil and gases, and improve hydrogen energy content within the synthetic petroleum liquids, which may be produced from a variety of different feedstock material sources.

Embodiments of the invention may provide a new and unique genre of pyrolysis, which may be characterized for the sake of convenience, and not by way of limitation, as a monolithic dome retort and (optionally) dome pyrolyzation, is the dome structure may be built or otherwise provided atop a surface as substantially an ellipse or half sphere infrastructure supported solely or primarily by one or more surface based footings, stem walls, floors or basement structures to create a large retort. The dome may provide a self-supported monolithic dome barrier, and may be created of any material including cement mixtures which have high temperature attributes to withstand the heat within the retort. In some embodiments, the dome may be in the form of a half sphere (i.e., a hemisphere) providing structural strength to maintain a shaft opening therein, and may serve as a barrier for that enables control of heat and vapors. The construction and use of a dome retort system may be made possible by using a large dome, then using conventional floor, auger and reclaimer technologies to discharge the organic material from the dome retort at, through or near the lower floor area of the dome retort. By utilizing a basement beneath the floor support supporting the retort dome area, mechanical drives, heating injection conduits, and lower level quenching and conveyance systems can heat, cool and transfer large volumes of organic material. A dome retort system may have dimensions from ten feet in diameter to well over 300 feet and can be constructed similarly in height. These dimensions, when combined with industrial strength associated volume augers and reclaimers modified to withstand the thermal and chemical conditions of the retort, may provide a relatively large pyrolysis treatment and retorting chamber. Nevertheless, the chambers may allow for at least substantially complete assurance of containment of hydrocarbon fluids and vapors and may be less unsightly relative to large steel factories. Aesthetically pleasing, these retorts may produce relatively larger volumes of oil that cannot be efficiently and economically achieved from the same feed materials otherwise in previously know retorts. The dome shell may be built using known air form, sand removal and geodesic dome construction methods. Such shells can be monolithic or modular, and can be constructed of materials for enhanced corrosion resistance, thermal control, vapor control and structural integrity. Using such a large-volume dome retort system, the residence time duration of the heated hydro carbonaceous material within the dome retort can be maintained for a period of days, requiring relatively lower temperatures in comparison to the higher temperatures employed in conventional, combustion based retort processing with in-retort residence times on the order of minutes, which higher temperatures create more emissions as well as a poorer quality of the resulting fuel products. By balancing scale, volume, residence time and capital costs, the promise and allure of fuels obtained from unconventional feedstock material using retorting processes is made possible.

Embodiments of the invention may avoid barrier subsidence and cracking issues associated with the prior art by incorporating the ellipse span integrity of an arch or dome over the heated containment, while enabling heating of a large, transported mass of feedstock material for hydrocarbon extraction, resulting in both high throughput and superior quality of extracted liquid hydrocarbon fuel. In at least one embodiment of the invention, the system is structured for substantially continuous feed of a large volume of feedstock material through processing to an exit. As a result, high spikes of produced liquid hydrocarbons associated with large, conventional batch processes are avoided, enabling the use of smaller tank farms to handle substantially continuous, more predictable volume liquid hydrocarbon production.

Furthermore, in some embodiments of the invention, implementation costs may be reduced as the dome spanning the process provides an isolation barrier for the system. Such a spanning dome may need to be manufactured only once due to the ongoing production of synthetic fuels from the hydrocarbonaceous material passing substantially continuously through the system the structural strength and integrity provided by a dome structure. Further, monolithic domes may have a relatively long useable life when compared to other structural configurations.

In one embodiment, the mechanical separation of feedstock material obtained from crushing may be used to create fine mesh size, highly permeable particles which enhance thermal dispersion rates into feedstock material passing through the dome retort treatment zone of the system. The increased permeability enables the use of low temperatures at long residence times while the particulate material continues to move (e.g., fall) through the system.

In one embodiment, one or more internal auger, reclaimer, or horizontal discharging systems may be employed to remove spent feedstock material from the dome retort once extracted hydrocarbons released from them have been collected.

In one embodiment, vertical heating or cooling conduits are fabricated and placed in appropriate geometric patterns hanging from the monolithic dome roof spanning over the piled feedstock material within the dome. These suspended heat transfer conduits work with the thermal heating system fluidly connected to a heat transfer fluid, in a preferably closed-loop, employing valve controlled junctions and heat transfer software for transferring heat into the feedstock material.

In one embodiment, refractory cement barriers, refractory barriers, steel barriers, clay, sand, gravel, liners, geo-membranes typical of engineered dome structures, or any combination of the foregoing, may be used to construct the substantially monolithic dome structure and associated shell creating the process isolation barrier and containment zone in which the hydrocarbon extraction process takes place.

In one embodiment, temperature and pressure sensors and monitoring mechanisms, fluid dispersion sensors may be provided and input to a computer controlled system with software to optimally control the aspects of the extraction process and manipulate varying gas and liquid extraction and injection (heated recycle gases) in connection with controlling the pass-through flow rate and temperature of hydrocarbonaceous material.

In one embodiment, insulation can be placed around an entirety of the dome, or selected portions of, the perimeter of the dome shell for optimized heat containment within the heated treatment zone to reduce required energy input for retorting, and also to protect the environment and human interactions from contact with the high temperature process heat.

In one embodiment, optimal geometric pipe placement for the introduction and recovery of heat energy by heat exchange from the moving, heated, processed feedstock material, may be placed within the lower half of the process isolation barrier, including embedded conduits conductively introducing heat or convectively injecting heat fluidly via reheated gas or vapor interaction with descending organic material prior to exit of the expended feedstock materials from the dome retort chamber.

In one embodiment, sectioned portions of the dome retort chamber may be constructed in alignment to enable gravity feed of hydrocarbonaceous material from upper sections to lower sections and ultimately exited out of the dome retort process chamber proximate the bottom thereof. In other words, feedstock material may be fed by gravity, assisted as necessary or desirable through the use of material transport elements such as, for example, augers, and reclaimers either situated vertically, at an angle or horizontally to channel, direct, force or pull heated organic material which may become difficult due to agglomeration. The throughput of such organic material may be conducted at a rate selected to optimize hydrocarbon extraction within the system. Temperatures and organic material residence times may be selected to optimize the quantity and/or quality of extracted hydrocarbons.

In one embodiment, various temperature zones can be created within the dome interior creating process isolation chambers separate from another for staged and sequenced heating methods, temperatures, gas, fluid and catalyst interactions and thermal transfers. Such interactions can be designed to crack longer chain hydrocarbons into lighter fractions (i.e., shorter chain hydrocarbons) within the pyrolyzing process or otherwise combine a portion of fluid or gas reactions within a chamber. This can include the disposition of high pressure chambers within the process isolation barrier to effect some refining or cracking within or below or adjacent to the dome retort. In such embodiments, the process can transfer liquids and fluids into and out of chambers, adjacent dome retorts which may be used for cooling, refining, quenching, preheating, and so forth. It is also contemplated that the use of basement rooms or tunnels beneath the dome retort floor will enable ready partitioning of various temperature zones, quenching, process water, slurry containments, oil containments, etc., so that distance and, therefore, piping and conduit costs can be reduced (e.g., mitigated). Underground chambers beneath the retort may be multiple chambers as well as vertical shafts or silos directly beneath or near the dome retort.

In one embodiment, a liner for the lateral perimeter of the process isolation barrier may be created with high temperature cements layered over rebar, steel mesh or wire reinforcements connected to bolts secured as a stem wall supporting a monolithic dome construction. Such stem walls can be excavated into the earth or provide a perimeter of the dome that may allow for the dome retort to be partially or substantially buried by soil, earth, aggregate or overburden. In such embodiments, it is envisioned that soil could be placed over the dome, and vegetative plants and trees could be planted in the soil over the dome such that the dome retort facility becomes more environmentally friendly and visually pleasing. Other liners, such as a fabricated steel liner, may be placed on the interior of the dome retort infrastructure using cemented and bolted reinforced liners, cables, etc. Free standing clay may be provided over at least a portion of the dome infrastructure to provide all improved thermal barrier and vapor barrier.

Monolithic dome retorts may be formed to comprise any of various materials including, but not limited to, sand, clay, gravel, volcanic ash, spent shale, cement, grout, reinforced cement, refractory cements, insulations, geo-membranes, steel liners, corrugated wall liners, shot-crete, refractory cement, high temperature epoxies, rebar, meshes, tension cables, air forms, geodesic frames, and the like. Drain pipes may be included within the dome structure, and such pipes may be insulated with thermally insulating materials. The dome retort shell may be used to contain at least substantially all vapor and liquids created within the treatment zone, and to simultaneously ensure that the outer environment does not interact with, or be contaminated by, operations conducted within, beneath, or inside of the dome retort itself In some embodiments, the dome structure may comprise a plurality of layers, each of which may comprise any of the above materials, and the layers may be disposed one on top of, and in contact with, the other. In other embodiments, one or more of the layers may be separated from others to create air, aggregate, insulation or other barrier containments between each. An outermost monolithic dome shell may serve as an insulating, vapor and thermal liner, and an innermost monolithic dome retort shell may serve as a barrier, and the two or more layer structures may collectively act as a dome retort structure.

In one embodiment, gravity assisted hydrocarbon material pass-through mechanisms as known in the art may be utilized to aggregate and channel interior introduction, pathways and exit of such material. Internal gases and fluids (including liquids and solvents) may also be handled and/or introduced by using internal pumping, channeling, condensing, heating, staging and discharging, collection, concentrating, piping, and drains, as known in the art. Gasses and fluids may be introduced into the retort through pipes or other conduits that may be embedded in the monolithic dome shell barrier itself or in its stem walls, basement walls, or its floor. Embodiments of fluid gravity drainage also include floor channels, riffles to guide and direct fluids, as well as reduce particle flow contained in the oil extracted from the feedstock materials.

In one embodiment, hydrocarbon materials of differing composition may be fed into the system for hydrocarbon extraction and exited therefrom through the gravity assisted movement of such materials in my mixed combination or grade or quality of coal, oil shale, tar sands, animal waste or biomass. Optimal compositions and layers or mixes of the foregoing may be introduced into the dome retort process area, and the system may enable different pass through movement rates, heating rates or residence times for each during the travel through the heated treatment zone. Liquids, chemicals, stabilizers, enzymes, solvents, or other catalysts may be used in any variety of ways in the extraction process to optimize or selectively create a desired chemical composition of the gases and fluids being created by heat and or the presence of, or lack thereof of pressure.

In one embodiment, sections within the gravity assisted dome retort chamber can be used to isolate materials, in absence of heat, or with intent of limited or controlled combustion or solvent application. Lower content hydrocarbon-bearing material may be useful as a combustion material and used solely for heating other hydrocarbon material passing through the system. In such embodiments, partitioned areas within the dome retort chamber may have oxygen selectively introduced to allow combustion, whereas simultaneously other areas may not have such oxygen or controlled combustion. One example of this may be a burner or other heat creating means such as a solid oxide fuel cell contained within the overall dome retort, which may be used to burn a carbonaceous material or hydrocarbon to create or radiate heat. In such instances, such burned material may also be gravity assisted and in a constant state of movement toward the bottom of the dome retort chamber and exit therefrom via a conveyor apparatus through an associated tunnel or other exit means to manage ash, char, charcoal or other by-products of the combustion process. Similarly, such isolated shafts within the dome retort chamber may contain heat transfer fluids, molten salt, or provide for exothermic chemical reactions to create heat or transfer heat to the passing hydrocarbonaceous materials within the system and in proximity to the heating shaft or conduit within or beneath the dome retort chamber.

In one embodiment, heat from the treatment zone, which rises to the top of the dome retort, may be redistributed back to the cooler areas of the bottom of the dome retort, creating convective currents along the dome interior wall lining or outside of the dome retort barrier to be introduced at a lower elevation. Such heat could also be transferred by internal conduits within discharge elevations internal to the dome retort chamber. Such internal conduits can collect, discharge or radiate any number of any type of gas, liquid, heat, or fluid transfer mediums. Heat from internal conduits may be originally derived from any heat source including, but not limited to, flameless combustors, resistance heaters, natural distributed combustors, nuclear energy, coal energy, fuel cells, solid oxide fuel cells, microwaves or any other type of fuel cell or solar or geothermically derived heat source. In the case of microwaves, internal microwaves could be used and interior dome shell surfaces can be constructed to guide, amplify or channel radiative heat energy into the organic materials passing through the dome retort.

In one embodiment, reducing agents such as hydrogen can be introduced to the dome retort treatment area under pressure and have a desired effect upon the liquids, gases and the hydrocarbonaceous material being processed. More specifically, so-called hydrotreating in the presence of a catalyst may be performed to a certain extent in an enclosed chamber within the dome retort shaft itself, adjacent to, or below the dome retort. Such hydrotreating could occur or be imposed to the feedstock material within the dome retort or to the hydrocarbon fluids collected from the feedstock materials. Pressurized chambers within the dome retort structure or proximate the dome retort structure may be pressurized (to pressures such as, for example, 2200-2300 psi) to release nitrogen, sulfur and other impurities from the extracted hydrocarbons, thereby increasing the quality of the extracted hydrocarbons for sale to market.

In one embodiment, the nature and quality of various fluid and gas compounds included in the extracted products can be altered prior to removal from the extraction system using, as an example, gas-induced pressurization. In such embodiments, pressure may also drive hydrocarbons from the heated organic material.

Aggregate, soil and sand placements external to the constructed dome retort structure can be used to structurally support the structure, thereby creating a thicker, insulating, and more reinforced perimeter liner of the dome retort chamber. Such aggregates may comprise Bentonite clay or mixtures thereof with spent shale, sand, gravel, aggregates, soil and/or volcanic ash. Such an insulative barrier may be equipped with moisture regulation mechanisms to replenish water driven off by the heat from the pyrolyzation process within such barriers on a constant or as-needed basis to maintain adequate moisture in the clay and associated materials. Other environmental monitoring layers and liners can be employed to track unwanted vapor escape. In such instances, tracking molecules dispersed within the dome retort during the process can serve as infrared markers or chemical markers to track and repair vapor or fluid escape.

In one embodiment, the monolithic dome structure itself can be repeated with a covering and encompassing larger dome made of any combination of materials, particularly those suited to form a heat and vapor barrier. In other words, the dome structure may comprise two dome structures including a larger dome positioned over a smaller dome. All dome monolith constructions can be placed on basement walls, footings, stem walls or combinations thereof.

In one embodiment, the heating rate for the hydrocarbon extraction process is controlled by selectively adjusting pressure, temperature, and chemical composition of introduced fluids and gases at different elevations within the dome retort structure. The redistribution of heat can be effected by heat exchangers removing heat toward the bottom of the dome retort and redistributing such heat back to preheater conduits suspended internally at the top of the retort dome proximate the substantially constant feed and gravity induced falling of the hydrocarbonaceous material. It is envisioned that temperatures of the vapor in such feed material zones could be at temperatures from 800 degrees to over 1,000 degrees F.

In one embodiment, within the dome retort, storage wells, high temperature pumps, gathering reservoirs, gathering pipes, slotted pipes, drains and tanks may be placed for collection of gases and liquids. Such tubular and non-tubular channels or conduits may contain catalysts as a packed bed within such containments for creating lighter fractions of hydrocarbon chains being extracted.

In one embodiment, heat within the dome retort may be introduced, controlled and manipulated by mechanical means at various elevations and sections or partitions within the dome retort.

In one embodiment, injected gases are controlled by pressure valves embedded within the floor of the dome retort. The floor may be substantially planar and purposely sloped to drainage and pump areas for removing gravity collected hydrocarbons. Such gas injection pressure valves may be embedded or recessed within the floor so as to allow heavy equipment such as front loaders, skidsteers and rubber tire machines to enter into the dome retort chamber to mechanically remove organic material without damage to such injection points. Buckets from such equipment then may be used to scoop organic materials without damage to pipes, drains, valves, or other equipment embedded within the floor of the dome retort structure.

In one embodiment, radio frequency (RF) mechanisms, solid oxide fuel cells, and other heating devices and emitters may be placed within an interior conduit extending throughout the dome retort vertically and may or may not be mechanically raised and lowered during heating of such devices in an effort to distribute or balance temperatures within the different elevations of the dome retort.

In one embodiment, pipes, drains, pumps, conduits and valves may be used to transfer, share, recover, redistribute and/or balance heat between sections or elevations of the dome retort structure, and/or to collect liquids and gases at various sections or elevations to avoid overheating or the need for liquids to migrate through spent organic material as it falls via the assistance of gravity within the system toward its exit via auger assisted discharge shafts. Internal baffles or other structures can be used to guide descending material to a plurality of circular rotating augers, horizontally positioned, to reclaim or push or pull the descending material to centralized, vapor sealed material discharge exits within the floor of the dome retort structure. The dome retort floor may include sloped sections on angles sufficient to channel gravity collected oil and liquids toward material discharge exits.

In one embodiment, augers and reclaimers are at or near the floor of the dome retort for moving the material to discharge points without damaging the floor. The floor itself may be flat, conical, sloped, or designed in any geometry so as to allow vertical augers, augers situated at angles to a conical funnel type floor, or completely flat to allow for use of horizontal augers. Embedded circular mechanical means may be embedded in the floors or side walls or even caverns within the floor or basement of the dome retort to support the outer reaches of such augers or to guide, rotate or push such devices. A dome retort can have one or more auger reclaimer systems. In the case of just one large horizontally placed reclaimer, outer walls of the dome retort may have an additional housing chamber for chain, bearing, or other mechanized motors or supports for the augers which encircle the dome retort floor surface and guide, support or enhance the performance of the augers. In such cases material may be pulled to internal shafts within the dome retort floor to flow in part by gravity, or they may be pushed to an outer wall chamber that allows organic materials to fall off the outer perimeter of the dome floor to a lower elevation comprising a conveyor or other conveyance means to an exit.

In one embodiment, computer assisted mining, mine planning, hauling, blasting, assay, loading, transport, placement, and dust control measures are utilized to continuously fill and optimize the speed and pass-through rate of mined or harvested hydrocarbonaceous material into and out of the dome retort. Following the exit of the spent hydrocarbonaceous material out of the lower portion of the dome retort floor, through, for example, a tunnel leading to an exit or quenching pool, such material can by conveyed to the surface via a conveyance system which controls off gassing from the material. It is envisioned that a heat quenching and gas squelching or suppressing technique be applied to the spent hydrocarbonaceous material upon exit of the spent hydrocarbonaceous material, or “char,” so as to enable its benign introduction to the open atmosphere and placement in a tailings management infrastructure, or clay or other liner impoundment.

In one embodiment, substantially precise measurement of weight of the hydrocarbonaceous material may be effected through use of truck or conveyor scales prior to feeding of the material into the dome retort for hydrocarbon extraction. Following extraction of hydrocarbon liquids by pyrolysis within the retort, as the hydrocarbonaceous material falls to its exit point, the depleted or spent material may be again weighed by conveyor weighing scales to acquire data or other information relating to extraction efficiency. Initially, hydrocarbonaceous material may be fed through a conveyor system which may have a means for preheating the material. The material is then fed through a vapor sealed charge feeder mounted atop the dome retort exterior surface, which may have excellent weight support capability for supporting other conveyor, dust control, vapor control, flare, piping and head house equipment. Following the gravity descent of the material down through the dome retort chamber to the chamber floor, horizontal or vertically rotating augers exit the organic materials to rotary, screw, vibrating or auger conveyors extending through, for example, a connecting tunnel. Computers may be used to control the monitoring, heat balancing, gas and fluid extraction measurement, chemical composition, flammability and or safety of such under dome retort tunnels and shafts used to exit the organic materials. Steam from quenching can be transferred as a heat transfer fluid to heat conduits within or embedded in the floor of the dome retort chamber. Water for steam may be constantly recycled and reintroduced to quenching zones.

In one embodiment, blasting, truck and shovel, haul truck transport and dozer leveling is contemplated for mining of hydrocarbonaceous feedstock material to be removed from an earth formation at high volume rates to feed the hydrocarbon extraction pyrolysis process within a dome retort. Comminuted organic material or harvest material may be fed to the dome retort by conveyor after sizing, sorting, crushing from standard mining methods.

In one embodiment, combustion of hydrocarbon material may be initiated toward the lower portions of the dome retort of the extraction system to create heat for pyrolysis of other hydrocarbonaceous material above such combustion zone within the process isolation barrier. Ash is removed from below, and such a combustion zone may be isolated from one or more heating (non oxygen) pyrolysis chambers within the dome retort. Oxygen can be injected into the combustion chamber.

In one embodiment, fluids can be introduced and circulated through the in-motion gravity falling hydrocarbonaceous material within the dome retort to rinse or reduce temperatures to modify various thermal or chemical states of the hydrocarbonaceous materials in process or post-process,

In one embodiment, sodium bi-carbonate and other mineral, precious metal and noble metal leaching solvents, including bioleaching agents, can be introduced within the constructed dome retort to extract metals and minerals from the hydrocarbonaceous materials, particularly, but not limited to, after hydrocarbon extraction, with or without thermal assistance, thereby extracting further valuable material from a feedstock material.

In one embodiment, core drilling, geological reserve analysis and assay modeling of a formation prior to blasting, mining and hauling (or at any time before, after or during such tasks) can serve as data input feeds into computer controlled mechanisms that operate software to identify optimal volumetric feed rates of a system or array of systems within respective dome retorts, and calibrated and cross referenced to desired production rate, heat rate, residence time or organic material composition to obtain improved yield or quality of produced liquid hydrocarbons. Example and non-limiting data inputs include pressurization of the dome retort, temperature of the dome retort, material feed input rates, preheating rates, material exit rates, auger speed due to specific gravity of a material, gas weight percentages, gas injection compositions, heating capacity, permeability of the falling hydrocarbonaceous material, material porosity, chemical and mineral composition, moisture content, and hydrocarbons per ton of material. Such analysis and determinations of desirable feed rates and mining rates may include other factors such as weather data factors such as temperature and moisture content impacting the overall performance of the hydrocarbons within the dome retort extraction system and its inputs and outputs. Other input data such as material moisture content, hydrocarbon richness, weight, mesh size, and mineral and geological composition may also be utilized as inputs to determine feed rate and optimum heat residence time, including factors relating to the economic efficiency of an extraction system comprising one or more dome retorts functioning alone or in tandem, each including a hydrocarbon extraction system, cooling system, pre-heating system or combinations thereof according to embodiments of the invention.

In one embodiment, mechanisms for treating extracted fluids and gases for the removal of fines and dust particles are envisioned. Separation of fines from extracted hydrocarbons falling by gravity to the dome retort floor can be a technical challenge. Within the retort, a concrete floor can be riffled or baffled horizontally against the drain flow direction of the sloping floor such that particles fall from suspension in the liquids and collect at the floor instead of flowing with the liquids to pumps, collection reservoirs and so forth. Cleaning methods or slurry pumps are envisioned to handle oil containing particles. It is envisioned that certain zones of the dome retort chamber could have entirely separate retorting chambers for small particles, thus allowing for the screening of material, but also allowing for the extraction of the screened material without interaction with larger particles or feedstock material. Screening can remove small particles before retorting, thereby reducing slurry problems within the oil. Once the oil is collected, other methods of filtering of particles can be employed such as, but not limited to, hot gas filtering, centrifuge separation and slurry decanting and liquid particle extraction from tanks or equipment situated beneath the dome retort in a basement-type containment or in, or in connection with, adjacent process room facilities.

In one embodiment, final sequestration of CO₂ produced by the heating within the dome retort or combustion therein or for any appurtenant upgrading or refining of the extracted liquid hydrocarbons, or for recycling processes, can be employed. CO₂ sequestration into existing or drilled natural gas or oil wells near the dome retort may be employed, and may be employed in concert with, or alternating with, water flooding.

Other methods of CO₂ sequestration may be utilized, where separate domes or spheres may be constructed using methods employing air forms and cementation dome construction to create tanks These tanks may be used for holding salt water or brine, which may induce solidification and settling of carbon dioxide into a slag that can be augered from such domes into a drying mechanism to create a cement admix. It is envisioned that the cement admix can be utilized in the cementation admixes of the monolithic dome retorts, monolithic dome tanks, monolithic dome buildings, or any other use of cement production including roads, curbs, sidewalks and so forth. Additional batch processing of cement may then remix such CO₂ sequestered cement admix with spent organic materials from the dome retort itself in various admixes depending on final use in construction or development.

In one embodiment, spent oil shale remaining in the dome retort, if oil shale is employed as feedstock material, may be utilized in the production of cement and aggregate products for use in the construction or stabilization of the dome retort monolith, footings, walls, stem walls, floors, roads, parking, fences, or to construct additional, adjacent embodiments of the aforementioned structures. Such cement products made with the spent shale may include, but are not limited to, mixtures that include one or more of Portland cement, calcium, volcanic ash, fly ash from coal, perlite, synthetic nano-carbons, sand, fiber glass, crushed glass, asphalt, tar, binding resins, cellulosic plant fibers, high temperature cement and epoxy.

In one embodiment, energy derived from alternative energy sources such as geothermal, solar, wind, wave, biofuels and algae farms may be used as an external heat source or to create heat for the extraction process. In the case of algae, algae carcasses may be themselves pyrolyzed in the dome retort to create a renewable biochar sequestration (negative carbon dioxide emissions) as a carbon sink to reverse global warming. Similar sequestration and retorting of biomass will also reduce carbon dioxide emissions.

In one embodiment, various stages of gaseous production may be manipulated through processes which raise or lower temperatures and adjust other inputs into the system to produce synthetic gases, which can include but are not limited to, carbon monoxide, hydrogen, hydrogen sulfide, hydrocarbons, ammonia, water, nitrogen or various combinations thereof.

In one embodiment, hydrocarbonaceous materials may be classified into various grades (such as, for example, hydrocarbon content or mesh size) and directed into various feedstock isolation shafts disposed within the dome retort chamber for separation of fines, or for high grading or low grading ore feeds within a portion of a chamber. Separate isolation chambers can have separate heating injection and/or separate oil collection (including for smaller particles which may create more of a slurry in terms of the gravity collected oil or hydrocarbons). Optimizing mixtures prior to or concurrently with introduction thereof into the treatment zone may have various chemical results desired for a given oil produced. For instance, different layers and depths of mined oil shale formations may be richer in certain depth pay zones as they are mined. Once, blasted, mined, shoveled and fed into a dome retort, richer oil bearing ores can be bundled or mixed by relative richness of hydrocarbon content with other lower grades for balancing or, for example, with coal or bituminous feeds such as tar sands or oil sands to add or subtract asphaltenes, residual oil or gas oil components of a desired crude oil chemistry. Optimal averaging of the hydrocarbon extraction process within a treatment zone may relate to a corresponding software designed to control the thermal heating rate or residence of such ore mixtures within the dome retort.

In one embodiment, CO₂ emissions from the dome retort extraction process may be recovered and used in Enhanced Oil Recovery oil fields, which may be adjacent to the dome retort according to an embodiment of the invention.

In one embodiment, injection, monitoring, recycle gas, heat transfer and production recovery conduits or extraction egresses may be incorporated into any pattern or placement within, under, around or penetrating the dome retort chamber.

In one embodiment, environmental monitoring wells underneath, around and beside the dome retort may be employed to monitor, collect or ensure performance that the dome retort containment has not been compromised. Tracer's can be monitored by infrared systems, and such systems can provide data in the environmental monitoring system.

In one embodiment, 3-D, thermal and feed rate software analysis and integrated data input and process simulation may be employed to predict the project economics and outcomes. Computers using software may employ design, operations, optimal extraction methods, and any related process to the extraction system.

In one embodiment, the associated mining or harvesting of hydrocarbonaceous material my dictate the placement and location of a dome retort and an appurtenant tunnel for the exit and proper conveyance and handling of spent hydrocarbonaceous material passes back to the surface for reclamation in proper tailings impoundments.

In one embodiment, surface support equipment such as condensers, pumps, hydrogen plants, gas handling units, electrical supply, heaters, data control, oil water separators, centrifuges, crushing, fines separation, slurry pumps, tank farms, vapor handling units, boilers, burners, recycle gas systems, pump houses, control rooms, monitoring systems, input and output computer housings for thermal couple data sensors and control valves, sensors and other reusable items may be truck mounted at the surface, electrically or fluidly connected to a dome retort or series of dome retorts.

In one embodiment, inner liners of the dome retort can be periodically replaced after a suitable time frame to protect pipes, internal hardware and the linings of the inside of the dome retort. A non limiting example is tar and stucco emulsions with shale or tar sands embedded in such liner coatings so as to repel abrasive wear on such surfaces.

In one embodiment, internal dome retort liners may wear out over time and be replaced at scheduled turnaround times, at which times all throughput for the extraction system is stopped for maintenance and repair of inner liners, pipes, and other system hardware. The use of tungsten carbide liners, hardfacing sprays, auger teeth, discs and mechanical parts and other wear protection elements and coatings may be used to protect surfaces in contact with falling hydrocarbonaceous materials, including but not limited to, materials handing chutes, channels, reclaimers, augers, sidewalls, doorways, and housings within the dome retort itself.

In one embodiment, processing of the liquids extracted by the dome retort may be effected to remove particles, nitrogen, sulfur, arsenic, other metals and add hydrogen under pressure. This process is known as “hydrotreating,” “hydrocracking” or “upgrading.” This process is optional and may or may not be employed to treat the hydrocarbon liquids extracted from the hydrocarbonaceous material.

In one embodiment, the pour point of extracted hydrocarbon liquid is lowered by manipulation of catalysts, pressure, temperature and injected gases, including, but not limited to, hydrogen.

In one embodiment, multiple domes are placed next to another each performing a different function such as pre-heating, retorting, cooling, quenching or other mineral extraction using solvents, leachates or other mineral and metal collecting liquid processes. Other processes performed in a retort may include rinsing.

In one embodiment, one or more vapor feed lock hoppers are situated on top of the dome and fed by sealed conveyors which may also have their own pre-heating and dust control mechanisms for heating the ore prior to entry into the greater dome. As ore is discharged, it may have any number of vapor sprays for initial flash heating, including such high temperature vapor contact to the particles while they are in suspension and falling into the chamber. It is envisioned that the dome retort ceiling can suspend chutes or pipes that serve as both dust control, particle protection (from falling and cracking to dust) and chutes or similar dust control injection means into the dome retort, which allow suspended vapors at high temperatures to interact with the particles falling by gravity or being lowered in a spiral fashion.

In one embodiment, instead of feeding the dome by gravity from above, the dome retort is fed by a screw lift rock pump through the floor of the dome retort via an intersecting basement tunnel conveyance. In this embodiment, a dome's conveyor and hardware could be completely protected on the interior of the dome.

In one embodiment, all auger discharge means may be reinforced with custom alloy materials that enable augers to withstand high pressure from ore resting on them above at very high temperatures. Any number of metal alloys specially designed for such strength, wear resistance, and temperature resistance is envisioned.

In one embodiment, the expensive aspects of distillation columns, hydrogen plants, tank farms, flares, cokers, sulphur recovery, nitrogen recovery, cracking units, boilers and vacuum distillation columns used in refining, coal to liquids flow diagrams, and hydrotreating or hydrocracking flow diagrams are shared with adjacent refinery complexes retrofitted or constructed to share vapors, heat, gases, hydrogen plants, and all other services, labor, maintenance, control systems, security, storage found within a refinery or a chemical or plastics plant.

In one embodiment, dome retorts are charged with ore that comprises coal, shale and tar sands (oil sands), catalysts, vacuum tower bottoms, tires, slurries, waste streams or any combination thereof. Such ore mixtures can provide additional hydrogen to another ore. For example, oil shale with approximately 13% hydrogen content may be blended with coal which contains approximately 5% hydrogen content to yield hydrocarbons of a higher quality.

In one embodiment, internal linings of stainless steel or any other material, including refractory materials, may be used to contain heat and prevent abrasion wear from passing, descending materials through the dome retort. These linings may be welded or bolted and may connect to the interior of the monolithic dome via steel plates or welding connections embedded in the cement of the dome retort shell on the interior side of the monolithic dome retort. A geodesic frame may be welded to these welding plates or welding connections for support of the overall interior liner. Behind the welded liner may be ceramic wool not affected by gases or vapors, since it is shielded by the welded, geodesic liner.

In one embodiment, following the construction of the air form dome crenosphere, an internal perimeter tunnel is constructed of concrete such that the dome then has a perimeter tunnel. Within the perimeter tunnel, a geared floor track may be laid encircling the dome on the floor of the perimeter tunnel within the concrete. Penetrating through the interior wall of the encircling wall may be a horizontal floor auger which pivots from the center of the dome retort to the tunnel wall, through the tunnel wall and down to a gear drive mechanically pulling itself in geared connection with the floor geared track. As the pivoting horizontal auger rotates, it pulls itself mechanically in tandem with the geared floor track in the perimeter tunnel. However, because the horizontal auger is piercing into the perimeter tunnel where the floor track is laid, and where an electric motorized gear shaft rotates or propels the gear along the track, the heated vapors from the dome chamber are isolated as the auger has a sealed vapor bearing which encircles the dome, separating the perimeter gear track tunnel. The gear track tunnel can be pressurized with inert gases such as Nitrogen and Carbon Dioxide, such that heat vapor and hydrocarbons cannot interact with possible sparks from the rotary motion of the encircling, floor mounted gear track, and its associated electric motors. It is envisioned that all such motors and gears may be sealed by vapor chambers or even fluidly submersed such that sparks cannot be created by the encircling gear shaft propelling in forward motion.

In one embodiment, an excavation is made such that an air form monolithic dome can be constructed within the excavation itself. Following the construction of the crenosphere dome retort, liners can be placed atop the dome. Then, one or more layers of gravel, sand, aggregates, etc., can be provided over the dome such that the dome then becomes subterranean. Within these layers, buried vapor recovery pipes and drain pipes may be laid so as to monitor for vapor leakage. Such aggregates and clays can be of a thickness sufficient to thermally insulate the dome. The layers can provide blast panels with sufficient embedded zones such that any internal explosion would blow off the blast panels avoiding significant surface blast of hardware atop the dome. A feedstock vapor sealed lock hopper is mounted to the buried dome providing access of organic material to be inserted in the dome. Organic material then is discharged in similar fashion through the dome retort flow via a tunnel connecting back to the surface.

In one embodiment, a remote controlled fire extinguishing system is deployed within the dome retort, around the dome retort, in the tunnels of the dome retort.

In one embodiment, thermal sensors are embedded in conduits vertically oriented within the dome retort to monitor the heat of the organic material and its heat rate as it descends to the floor auger system.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Summary does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 is a schematic side sectional elevation of an embodiment of a dome retort hydrocarbon extraction system, including a monolithic dome retort shell, footing, stem wall and underground basement chambers, tunnels, conveyors leading to impoundments and associated storage and processing systems of liquids for various uses according to an embodiment of the invention;

FIG. 2 is a side elevation of the monolithic dome retort with a view below the surface to tunnel exits according to an embodiment of the invention;

FIG. 3 is a side elevation schematic of a dome, which is cut away to reveal its dome retort floor, auger systems and associated discharge shafts as well as basement tunnels beneath the dome retort according to an embodiment of the invention;

FIG. 4 is a side cut away elevation of the dome retort showing the organic material piled within the dome retort according to an embodiment of the invention;

FIG. 5 is a top elevation of the dome retort interior showing a plurality of floor reclaimers extending from a plurality of pivot points according to an embodiment of the invention;

FIG. 6 is a top view elevation of the dome retort floor's heat transfer piping layout which may be used for recycle gas injection through the floor or for heating radiation from the floor itself according to an embodiment of the invention;

FIG. 7 is a top view elevation depicting the heating pipes within or atop the floor also showing the floor reclaimers circular patterns as well as a top view of the floor auger pivot systems according to an embodiment of the invention;

FIG. 8 is a top view elevation looking through the dome retort above to a below tunnel system which contains sealed vapor auger systems, quenching systems and conveyance systems according to an embodiment of the invention;

FIG. 9 is a side elevation of a cut away floor revealing embedded recycle gas injection valves in the floor of the dome retort beneath the circling floor auger system and discharge pivots according to an embodiment of the invention;

FIG. 10 is a side elevation of circular patterns above heat transfer pipes within the dome retort floor structure according to an embodiment of the invention;

FIG. 11 is a top and side elevation of a sample floor reclaimer auger leading to a sealed vapor discharge system. The auger system also shown represents an encircling track for propulsion of the auger embedded in the floor for various uses according to an embodiment of the invention;

FIG. 12 is a side elevation of a sample floor reclaimer system and propulsion track leading to a vapor sealed discharge auger for various uses according to an embodiment of the invention;

FIG. 13 is a side elevation of a sample dome retort tunnel beneath the dome retort floor representing in this diagram a discharge point in the process flow to a spring activated quenching unit leading to a sealed conveyor system for various uses according to an embodiment of the invention;

FIG. 14 is a side elevation of a sample dome retort sealed feed conveyor leading to a sealed vapor lock hopper resting atop the dome for various uses according to an embodiment of the invention;

FIG. 15 is a side elevation of a multiple dome retorts connected in a process flow arrangement for various uses according to an embodiment of the invention;

FIG. 16 is a side elevation of a sample dome retort showing a multiple of coned extraction zones leading to a vertically oriented auger system connected to tunnels below for various uses according to an embodiment of the invention;

FIG. 17 is a side elevation of a sample dome retort configuration whereby floor auger circling layout have interior slope floors which guide materials to those circling auger patterns for various uses according to an embodiment of the invention;

FIG. 18 is a side elevation of a sample dome retort cone and vertical auger configuration with associated conduit piping for various uses according to an embodiment of the invention;

FIG. 19 is a top elevation of a sample dome retort cone floor with associated recycle gas injection points for various uses according to an embodiment of the invention;

FIG. 20 is a side elevation cut away of a dome buried within a formation in this embodiment. Layers of aggregate and clay are depicted as well us an underground tunnel whereby processed organic material may be exited from beneath the dome retort;

FIG. 21 is a side elevation of a dome retort wherein in this embodiment, a close up view of a perimeter tunnel room provides a clean room for a perimeter anger track and its motor drive unit. The perimeter tunnel room atmosphere may be made inert by sustaining injected inert gases such as carbon dioxide or nitrogen. This drawing depicts a rotary bearing and sliding perimeter wall which houses the rotary sealed auger bearing;

FIG. 22 is a side elevation of a dome retort wherein in this embodiment, a perimeter tunnel room provides a clean room for a perimeter auger track and its motor drive unit. The perimeter tunnel room atmosphere may be made inert by sustaining injected inert gases such as carbon dioxide or nitrogen; and

FIG. 23 is an internal geodesic frame bolted to the internal side of the dome retort comprising an internal frame which may provide support for a steel liner or other liner for various uses within an embodiment of the invention.

The drawings are not necessarily to scale.

### DETAILED DESCRIPTION

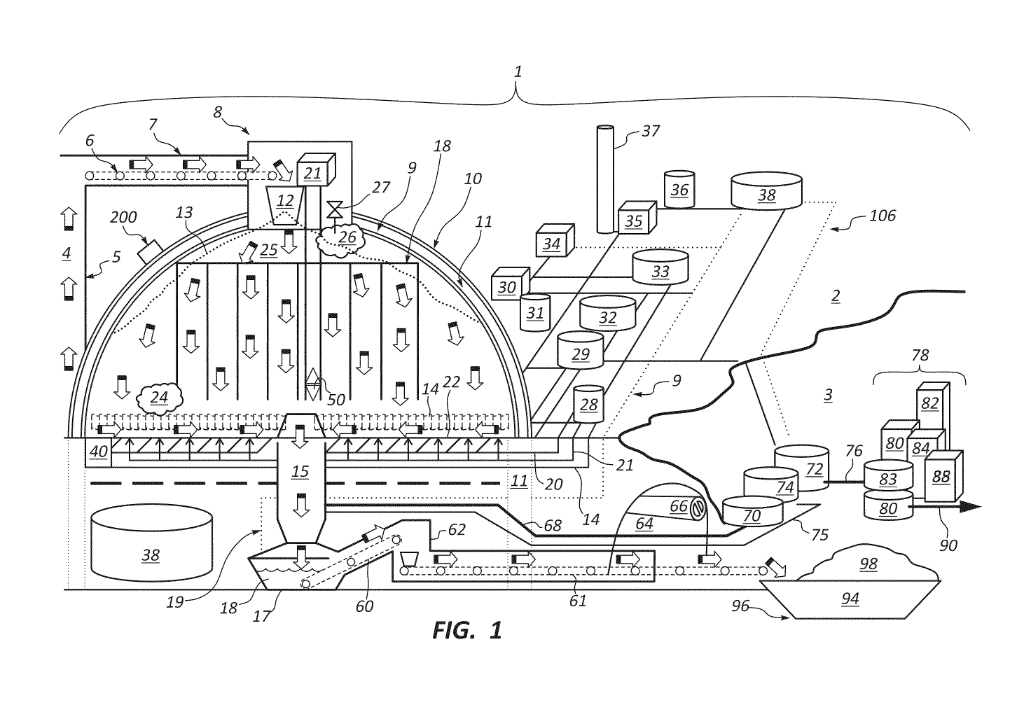

FIG. 1 is a cutaway schematic side elevation of a dome retort system 1 on a surface 2 atop an earth formation bluff 3. Organic material 4 is elevated by conveyor elevator 5, to a horizontal top conveyor 6, sealed by a vapor sealing means 7, and conveyed into a connecting head house atop a dome retort structure 9, with a multiwall dome shell layer 10.

Organic material 4, is fed into a vapor sealed lock hopper 12, also contained within head house 8, and upon sealing the vapors 24 within the dome retort 9, feeds organic material until a large permeable body of organic material is piled 25 within the dome retort 9.

Due to the constant rotation of a center pivoted floor auger system 14, piled organic material 25 slowly descends over a given time through the dome retort 9. Hot gases 24 are injected through the dome retort floor 22, allowing for direct particle gas interaction with pile of permeable organic material 25. Additional heat radiates from dome retort floor 22, which is heated below by a heat transfer piping system 20 as well as by heat from a hot recycle gas system 21. Heat rises through the organic material permeable body 25, which causes pyrolysis, creating additional hydrocarbon gases 26 extracted from the organic material 25. Hydrocarbons may be collected with the assistance of gravity into tank 40, which may be positioned toward a low end of the floor 22 which may be sloped.

As floor reclaimer and auger system 14 rotates, it spirals its augers, thus pulling organic material toward a vapor sealed discharge auger 15 that is situated above a quenching and cooling unit 16. Steam is created in the quenching process and flows to an oil water separator 28 and may be recycled into the floor heat transfer system of conduits 20. Oil collected from oil water separator 28 is sent to oil tank 38 and water is recycled back to quench system 17 as quench water 18.

Additional heat may be provided to dome retort 9, via an internal heating delivery conduit 21, which may be comprised of a fuel cell, combustor or other heating element at position 50. Internal heating delivery conduit 21 may also house a vapor recovery system leading to condenser 29.

An excavated shaft entrance 64 leads to a tunnel beneath dome retort 9 and its dome retort floor 22. Within tunnel 64 are oil collection pipes 68 and nitrogen or inert gas pipes 75 leading to the dome retort 9. Also exiting the tunnel 64 is conveyor system 60 which conveys spent organic material exiting quench system 17 and said conveyor system is vapor sealed to collect steam and gases by conveyor ventilation hood 62 leading to exiting conveyor system 61 which may be portable conveyor leading to a tailings impoundment 94 with a permeability control liner 96 covered by reclaimed landscape soil 98 for long term impoundment of spent organic materials.

The dome retort structure defined by shells 9 and 10 may have a dimension, by way of example, of from 10 to 400 feet in diameter and up to greater than 200 feet in height. Dome retort shells 9 and 10 may have a separation zone or distance 11 therebetween, which helps provide thermal containment. Stem wall or footing 11 may be of any height and the dome retort structure defined by shells 9 and 10 may be connected by rebar reinforcement (not shown). It is contemplated that the height of any stem wall may be any height and the connecting dome retort structure 9 and 10 may be of any diameter serving as a cap. Similarly, the dome retort floor 22, may be constructed of any diameter.

Vapor recovery exit 27 pulls vapors 26 from the dome retort 9 into the recycle gas system leading to the condenser 28. Non condensed vapors can be burned in burner 30 to provide make up heat into additional non condensed vapors used as a heat carrier through the hot recycle gas system 21 and heat transfer conduit system 20.

The condensed oil tank 38 may be connected via a pipeline 106 to be combined with produced oil removed from tunnel 64 via gravity-collected oil pipeline 68 and stored in oil tanks 72 for additional storage, or transported elsewhere as desired. The non-condensable hydrocarbons collected in the vapor recovery pipes (not shown) may alternately be sent for sulfur removal in the gas clean up unit 35. In certain embodiments, non condensed excess gas may be flared via a flare 37. Cleaned gas from the gas clean up unit 35 may be burned in burner/boiler 30 as a heat source for retorting heat within dome retort 9, or may be used for other process needs, delivered into dome retort 9 by the down hole heat delivery shaft 21, or delivered in a heated state via the recycle gas injection system 21 as a hot recycle gas 24 which rises through organic material 25 to be collected at vapor recovery exit 27. Excess gas from burner/boiler 30 may, optionally, be flared via flare stack 37 or transported to market or utilized in a power generator 35 which may also be fueled by fuel tank 36.

Direct heat delivered by the down hole heat delivery shaft 21 will augment heat being provided to the dome retort 9 by other heat sources lowered down separate conduits within the down hole heat delivery shaft 21. Other heat delivery means lowered down the down hole heat delivery shaft 21 may include, but are not limited to, solid oxide fuel cells, microwave generators, electric resistance heaters, down hole combustion burners and any other heat delivery means located substantially in the vicinity of position shown as 50.

Piled organic material 25 introduced into the dome retort 9 substantially continuously descends through the dome retort 9, removed by the augers 14 or other material handling mechanisms. Gravity pulls the organic material 25 to the dome retort floor 22. The dome retort 9 interior maintains thermal, vapor and pressure differences between tunnel 64. Tunnel 64 and head house 8 may be sealed and pressurized with nitrogen or carbon dioxide gases and maintained at a higher pressure than the dome retort 9 so as to isolate hydrocarbon vapors from areas where mechanical devices and electrical devices are operating in regards to pumps, augers, conveyors and the like.

Within the dome retort 9, organic material 25 under gravity as direct gas-to-particle heating occurs with rising heated recycle gas 24, heat from down hole heater shaft 21 radiates or is directly delivered into the retorting chamber 9 at various shaft elevations, including from lowered heating means positioned at 50. After heating in retorting chamber 9, organic material 25 descends into vapor-sealed lock hopper 15. The floor mounted lock hopper 15 intersects the floor 22 and maintains thermal, vapor and pressure differences between quench chambers 17 and dome retort chamber 9 such that retorting chamber 9 (with more hydrocarbon vapors) may be at a lower pressure than quenching chamber 17 so as to prevent vapor or thermal communication from one chamber to another, yet allow organic material 25 to descend on a substantially continuous basis.

Steam generated from the relatively hot, spent organic material 25 contacting the quench water 18 within the quench system 17 can be transferred as a heat transfer fluid via thermal transfer conduits 20 underneath the dome retort floor 22 or via steam vapor recovery exit 19 as desired. The quenching chamber charge feeder 15 keeps thermal, vapor and pressure differences between chambers 17 and 9 separate. It should be understood that vapor-sealed charge feeders 12 and 15 are of designs configured to seal vapor, collect gravity-draining oil and liquids as well as slurries, particles and fines. Particle-containing oil and slurry is pumped from these locations and from floor drain 40 via gravity-collected oil pipe 68 and exits tunnel 64 to oil/water separator 70 and then to oil tank 72. Nitrogen generator 74 may be used to generate inert nitrogen gas to be delivered by nitrogen gas pipe 75 for oxygen purging of the tunnel, the lock hopper 15 and 12 or within the dome retort 9 itself. It may also be used for cooling in one or more contained mechanical housings in such areas.

The retorted, spent organic materials 25 quenched by quench water 18 are conveyed on conveyor 60 and 61 through tunnel 64 with tunnel ventilation system 66 providing fresh air to the tunnel if not under inert conditions during operations. Conveyor hood vent 62, which captures and channels any remaining off gassing from organic materials 25 on conveyor 61 can be sent to oil water separator and through vapor recovery unit 29. As organic materials 25 exit tunnel 64 on conveyor 61, a series of mobile or fixed conveyors (or trucks not shown) can convey or haul spent tailings (used organic material) to tailing impoundment 94 with permeability control infrastructure 96 made of any material or combination of materials, and be covered and reclaimed by top soil 98 and re-vegetated. The combination of impoundment, liner, and top soil may or may not include a lining of compacted Bentonite clay and may include drainage pipes (not shown) to divert water from said tailings impoundments.

The collected gravity oil in tank 72 can be sent by pipeline 76 to a separate or adjacent refinery and upgrader 78. The refinery/upgrader includes, but is not limited to, process equipment including a hydrogen plant 80, a distillation tower 82, a hydro-treater 84, arsenic removal means 83, and nitrogen removal and handling means 88. Further to a refinery are other cracking and reforming processes (not shown) for the production of gasoline. Following upgrading or refining at such a facility, the liquids have improved energy, near zero sulfur and nitrogen content and are ready for shipping to oil and/or fuel markets via pipeline 90. Hydrogen plant 80 can send hydrogen via hydrogen pipeline 81 as a fuel to a solid oxide fuel cell lowered down hole heater shaft 21 or provide hydrogen for power generation to a fuel cell within power generator 36 to power all process needs. Additionally, hydrogen plant 80 can provide hydrogen to the dome retort itself injected as a hydrogen donor in the pyrolytic process. Other donors of hydrogen can include bottoms from 82 or other residual refinery coke or bottoms from adjacent processing to the dome retort 9.

At least one blast panel 200 may be placed within the dome retort 9 structure to provide blast pressure relief in case of an explosion or other rapid and/or uncontrolled combustion rate.

Carbon dioxide from dome retort system 1, combustion burner/boiler 30, refinery 78, hydrogen plant 80 and so forth can be collected via carbon dioxide management system (not shown) and injected into a well bore (also not shown) as geologically sequestered carbon dioxide in the formation 3.

To start the heating process for hydrocarbon extraction, propane or other fuel storage 36, supplies fuel to burner/boiler 30 and to power supply generator 35 for all process boilers 30, blowers (not shown), pumps (not shown), conveyors 61 and 6. As retorting occurs within the dome retort system 1, some collected hydrocarbons from the retorting process may be used to provide make up fuel and also act as a heat transfer fluid.

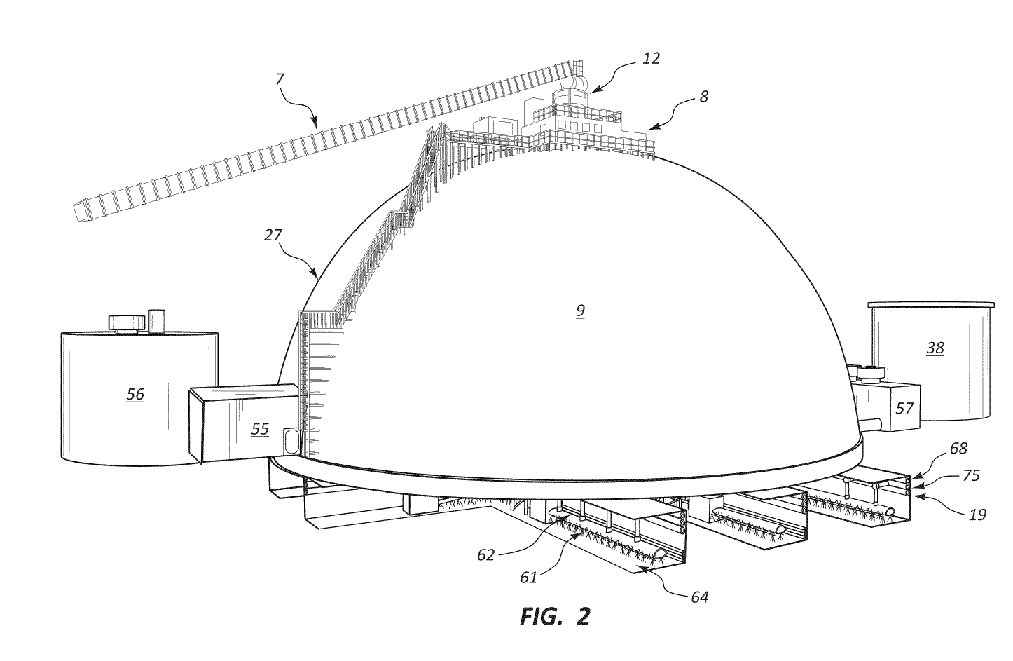

FIG. 2 shows a three-dimensional side elevation of the exterior of a dome retort 9. Sealed conveyor system 7 feeds organic material into lock hopper 12 on top of head house 8. Vapor recovery pipe 27 fluidly conveys vapor to vapor handling system 55. Recycle gas can be heated in gas heater 30. Multiple tunnels 64 exit from beneath dome retort 9 containing conveyors 61 sealed by conveyor vapor hood 62. Gravity collected oil pipe 68 and inert gas piping 75 are exited from tunnel 64 along with steam recovery piping 19. Crude oil tank 38 and gas recycle compressor 57 is also shown in this particular embodiment.

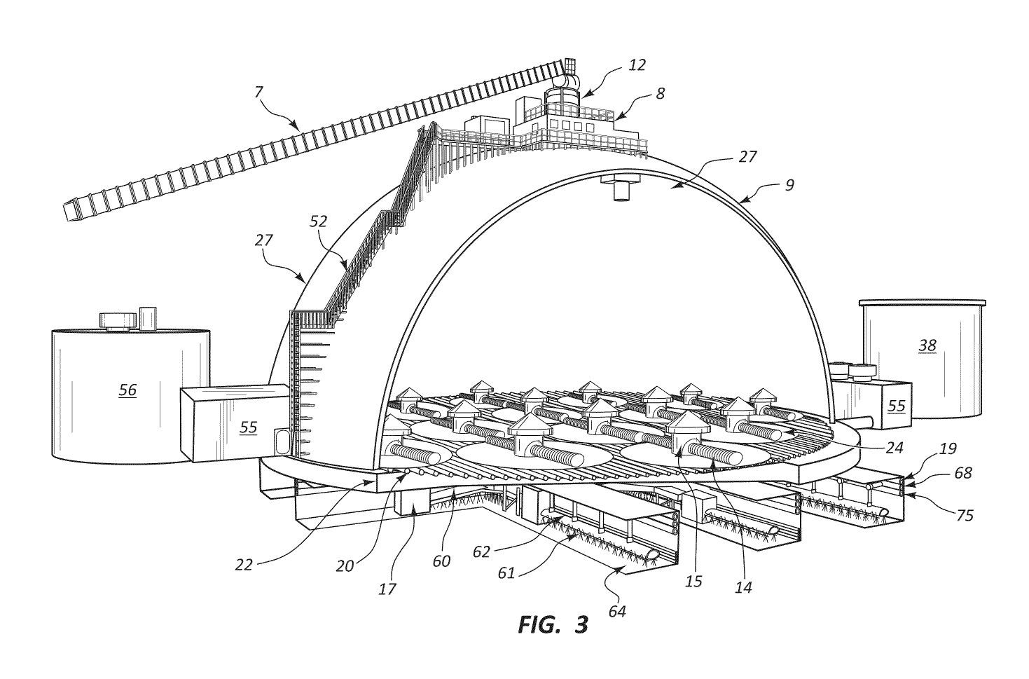

FIG. 3 shows a three-dimensional side elevation of the exterior and interior of a dome retort 9. Sealed conveyor system 7 feeds organic material into lock hopper 12 on top of head house 8. Vapor recovery pipe 27 fluidly conveys vapor to vapor handling system 55. Recycle gas can be heated in gas heater 30. Multiple tunnels 64 exit from beneath dome retort 9 containing conveyors 61 and 60 sealed by conveyor vapor hood 62. Quench system 17 is discharged by conveyor 60. Dome retort floor 22 is heated by heat transfer conduits 20 and floor embedded hot recycle gas conduits 21. Gravity collected oil pipe 68 and inert gas piping 75 are exited from tunnel 64 along with steam recovery piping 19. Center pivoting vapor sealed ore discharge unit 15 houses horizontal floor reclaimers and augers 14. Crude oil tank 38 and gas recycle compressor 57 are also shown in this particular embodiment.

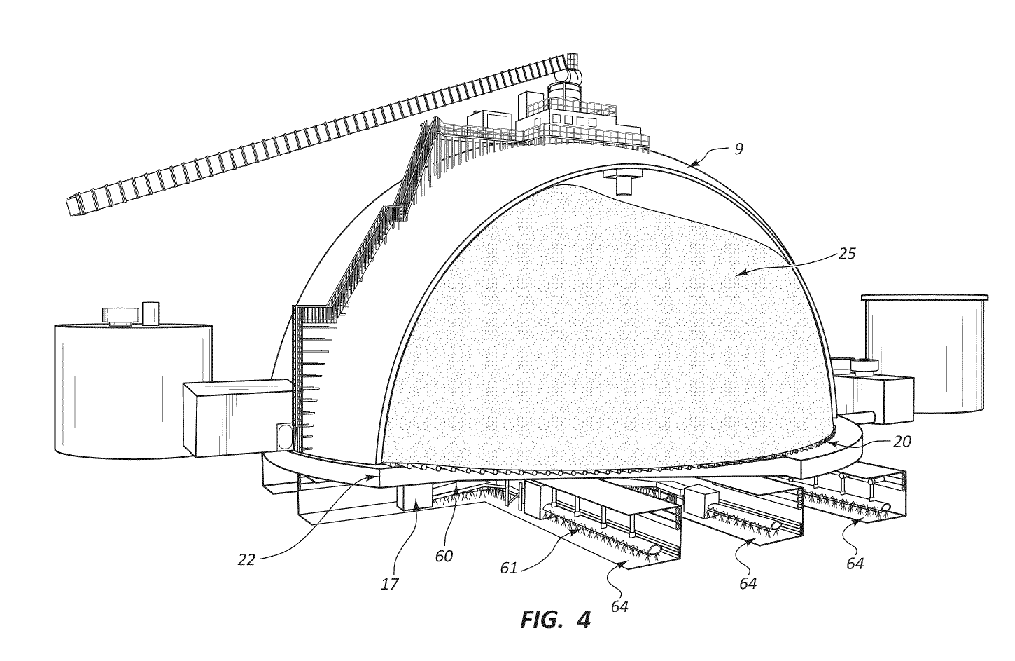

FIG. 4 shows a three-dimensional cut away rendering of the dome retort 9 of FIG. 3 filled with organic material 25 being heated by heated floor 22 receiving heat from heat transfer pipes 20. Once hydrocarbons are collected from organic material 25, the organic material is exited to quench system 17 and conveyors 60 and 61 from exiting tunnel 64.

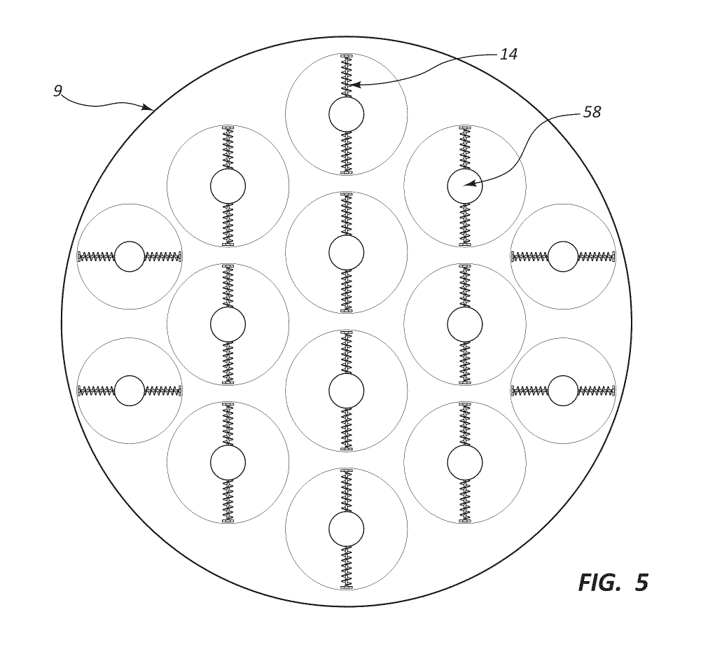

FIG. 5 shows a top view from inside dome retort 9 looking down at auger systems 14 and their center pivoting auger housing 58 in one embodiment of the invention. Each auger is rotated about its longitudinal axis, and each auger may be pivoted about its respective housing 58 such that materials overlying the circular areas illustrated in FIG. 5 are eventually collected by the auger systems 14.

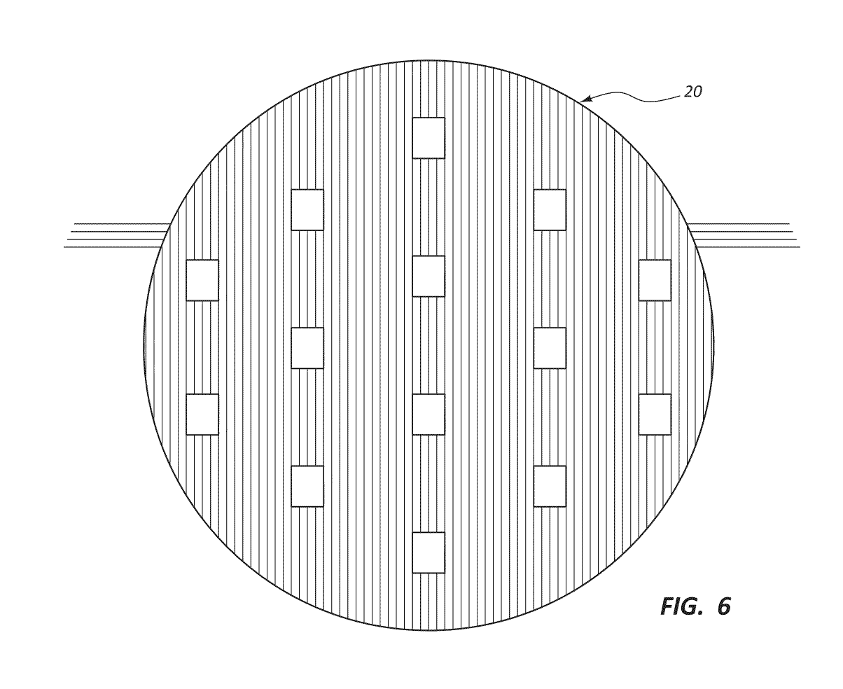

FIG. 6 shows one particular embodiment of embedded pipe conduit system 20 within the floor of the dome retort. As shown, substantially the entire floor of the dome retort structure may be heated using the embedded pipe conduit system 20.

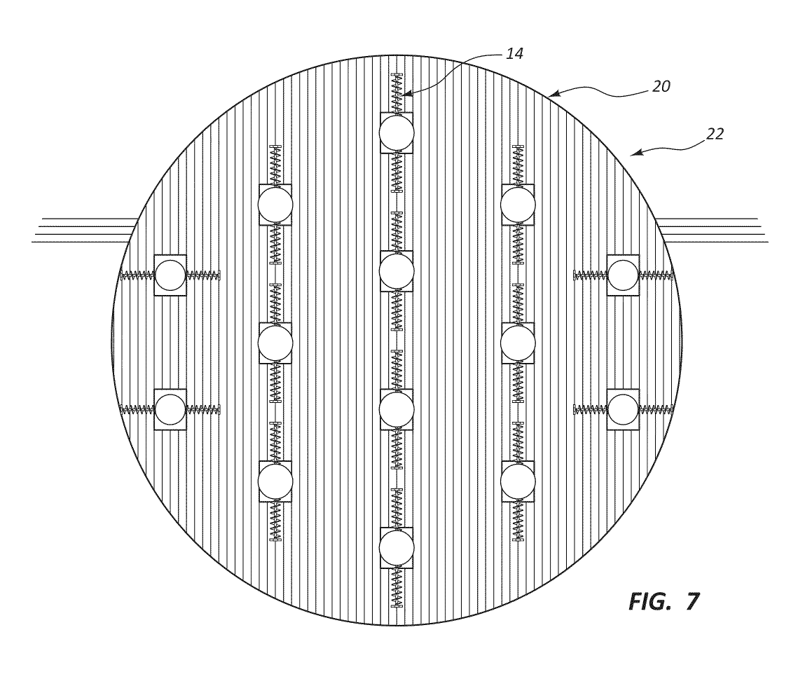

FIG. 7 shows one particular embodiment of embedded pipe conduit system 20 within the floor of the dome retort 22 with circular floor auger system 14 above.

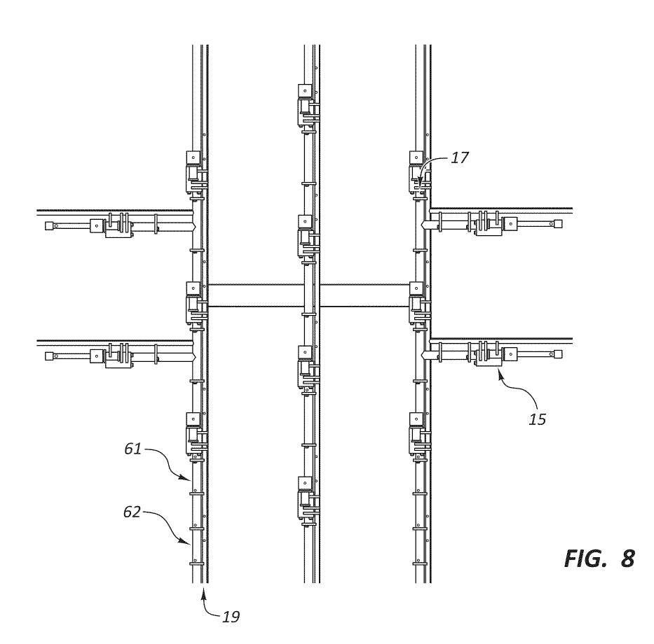

FIG. 8 shows a top view of the basement tunnels 64 of the dome retort. Multiple tunnels 64 can be configured to provide exit pathways for conveyors 61 with conveyor hoods 62 fluidly connected to steam recovery pipe 19. Vapor sealed lock hopper 15 functions adjacent to quench systems 17 which cool the organic material as it is discharged from the dome retort structure.

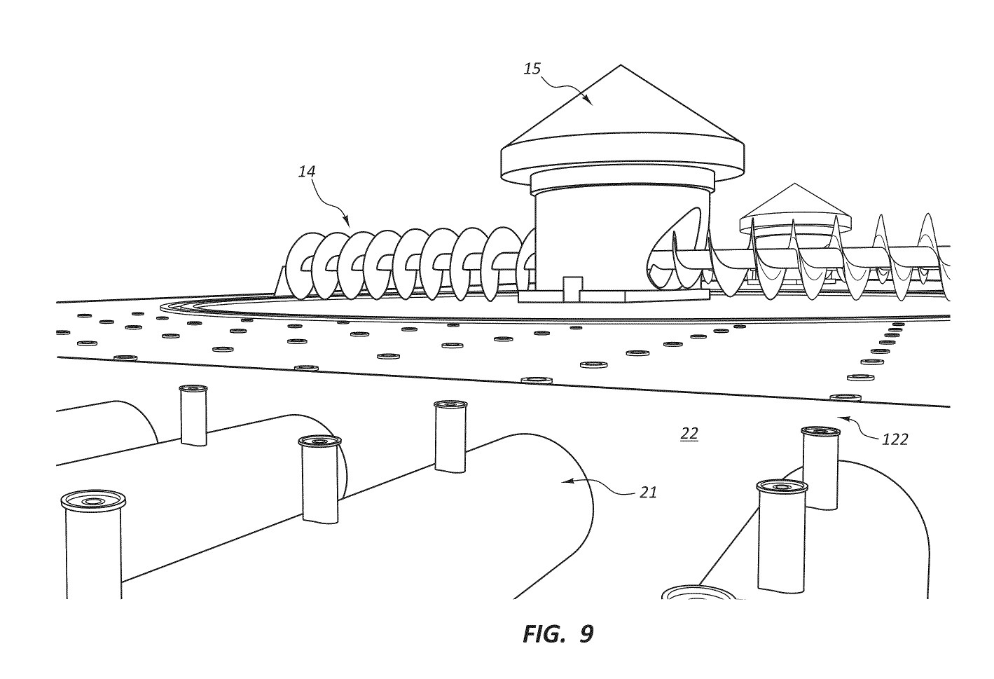

FIG. 9 shows a three-dimensional cut away of the dome retort floor 22 which contains embedded pressurized vapor injection valves 122 fluidly connected to hot recycle gas piping system 21.

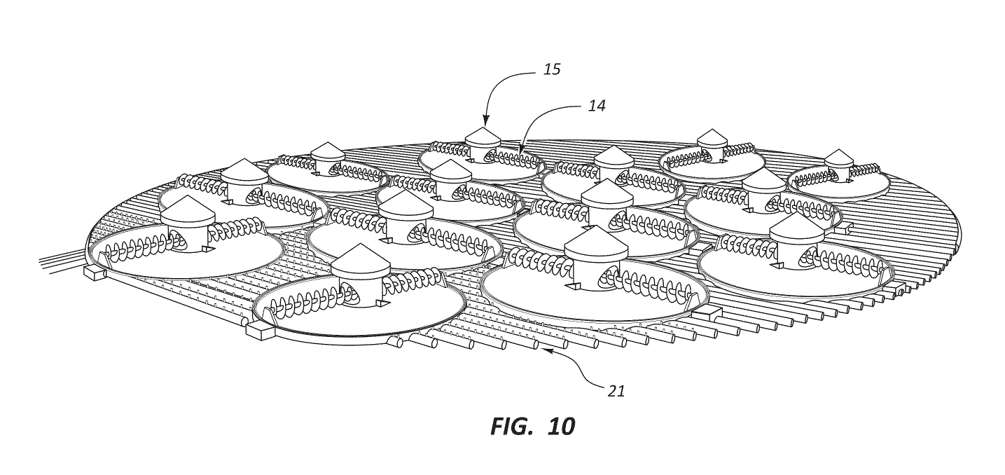

FIG. 10 is a three-dimensional view of the hot recycle gas conduit system 21 beneath the floor of the dome retort 22. Vapor sealed lock hopper and auger housing 15 powers and turns floor auger reclaimers 14.

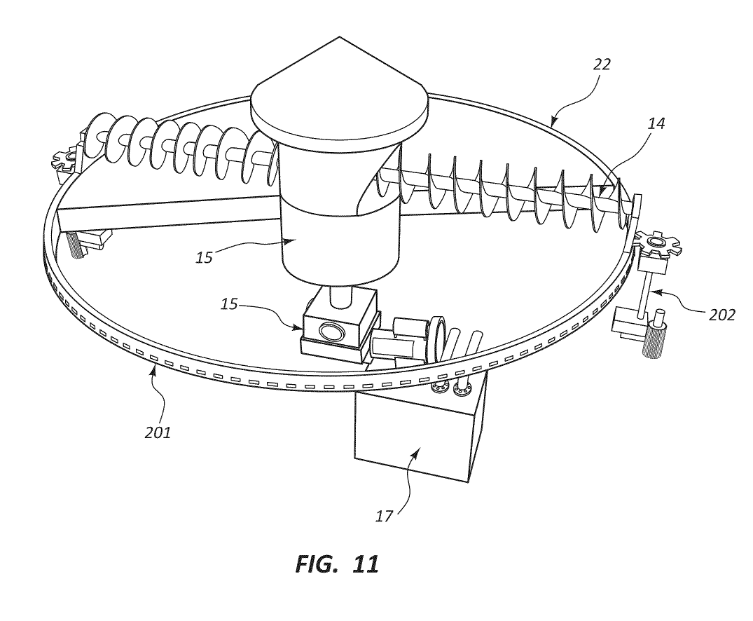

FIG. 11 is a three-dimensional view of the dome retort floor 22 which is cut away, the auger 14, the vapor sealed lock hopper 15 beneath the floor 22 which leads to the quenching chamber 17 below. Also shown in this particular embodiment is a circular auger track 201 with auger track propulsion gem” motor 202.

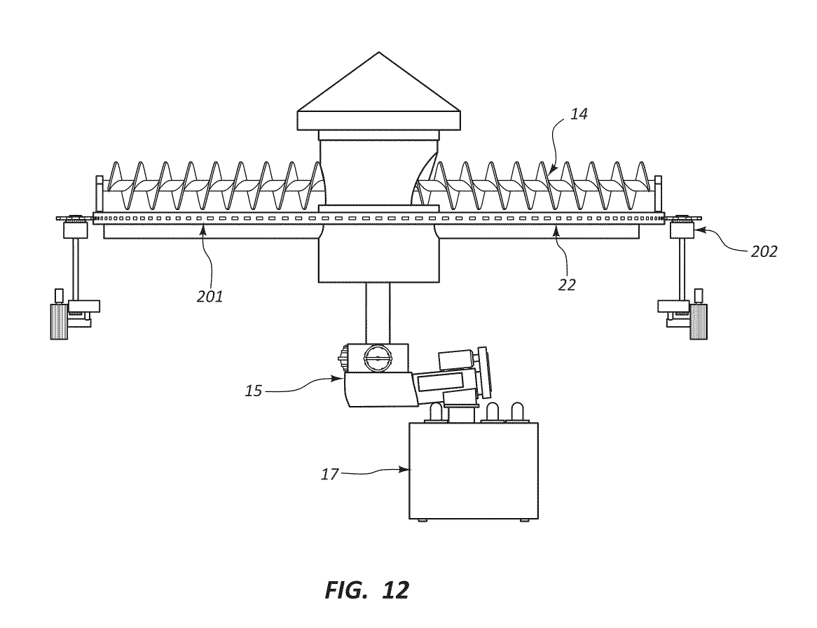

FIG. 12 is a side view of a three-dimensional view of the dome retort floor 22, the horizontal floor auger 14, the vapor sealed lock hopper 15 beneath the floor 22 which leads to the quenching chamber 17 below. Also shown in this particular embodiment is a circular auger track 201 with auger track propulsion gear motor 202, which may be used to drive the pivoting movement of the auger 14 about the auger housing 58.

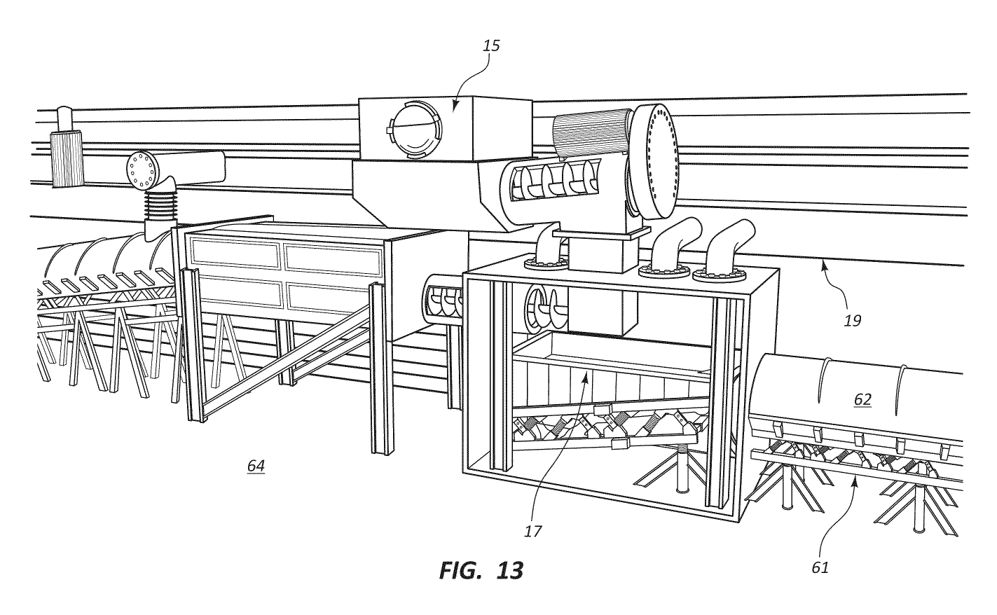

FIG. 13 shows a three-dimensional side view of equipment within the underground tunnel 64 beneath the dome retort above. Vapor sealed lock hopper 15 exits organic material by sealed auger system into quench containment 17 which is discharged by conveyor 61 sealed by conveyor vapor control hood 62 which can be fluidly connected to recover steam and vapors via steam and vapor pipe 19.

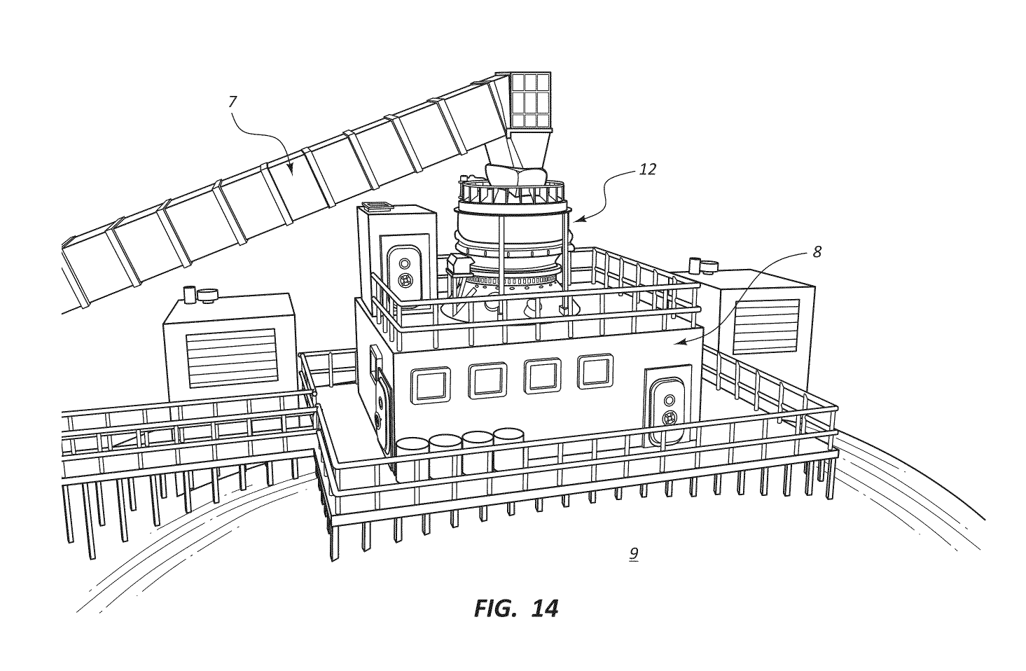

FIG. 14 shows the top of the dome retort 9 supporting a head house 8 which also supports a sealed vapor lock hopper / charge feeder 12 connected to a sealed conveyor system 7.

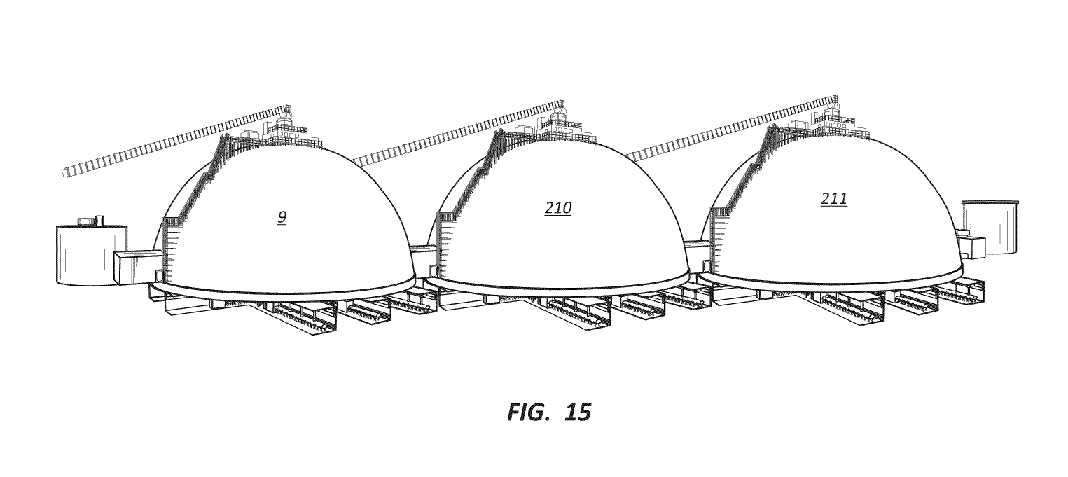

FIG. 15 shows a side elevation of multiple dome retorts that may be utilized in tandem together as part of a retorting system. Dome retort 9 may comprise a preheater dome. Middle dome retort 210 provides primary retorting and hydrocarbon recovery and a cooling dome 211 is shown in this particular embodiment. It should be understood that all conveyor, pipe, controls, systems, processes, tanks and tunnel systems could comprise any number of connected configurations.

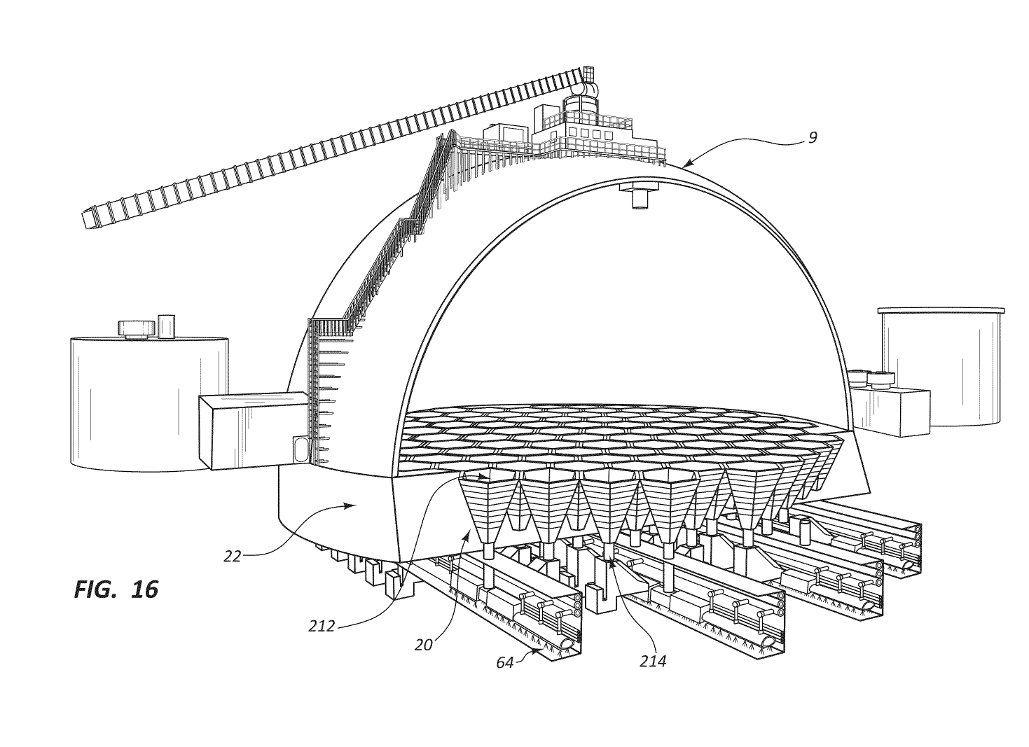

FIG. 16 shows another embodiment of a dome retort 9, wherein the floor of the dome retort 22, is comprised of sloped cones 212, which direct organic material to vertical augers 214. Said sloped cones 212, are lined with heating conduits 20.

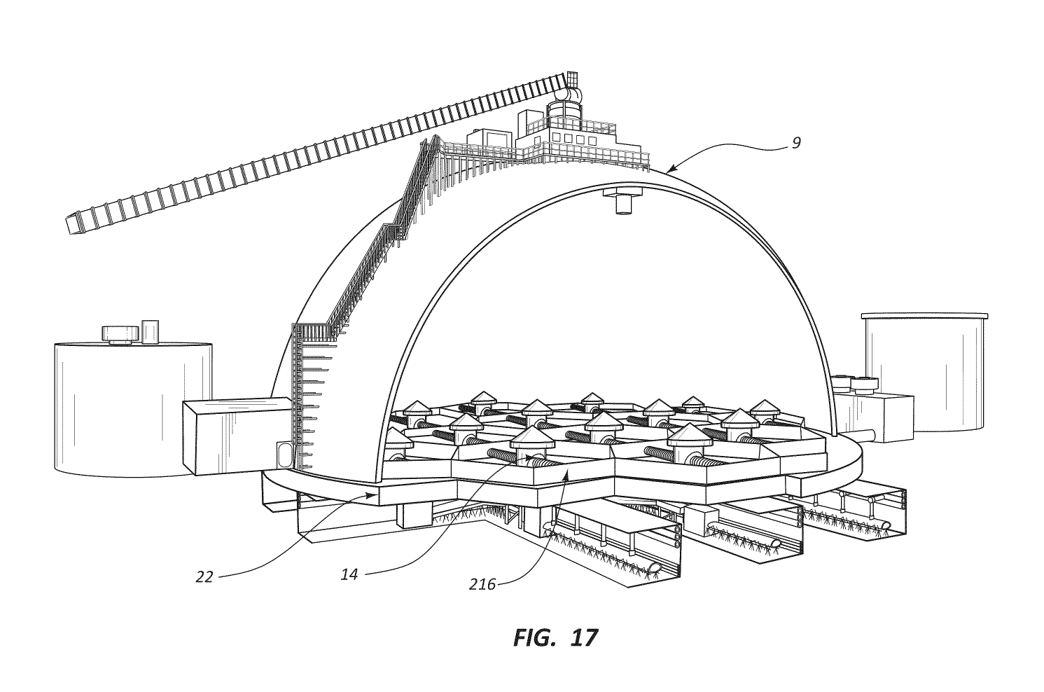

FIG. 17 shows an embodiment of the invention wherein the dome retort floor 22 additionally is comprised of sloped structures 216 which guide and direct organic material towards floor auger and reclaimers 14.

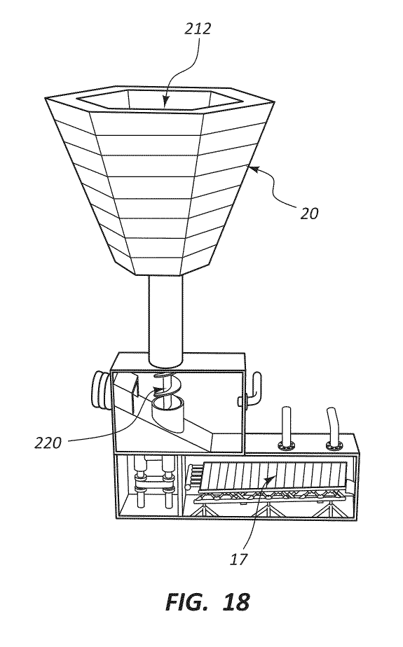

FIG. 18 shows a three-dimensional side elevation of a particular embodiment of the invention wherein a sloped cone floor component 212 guides organic materials toward a vertical auger reclaimer 220, exiting said organic material into a quench basin 17 for cooling.

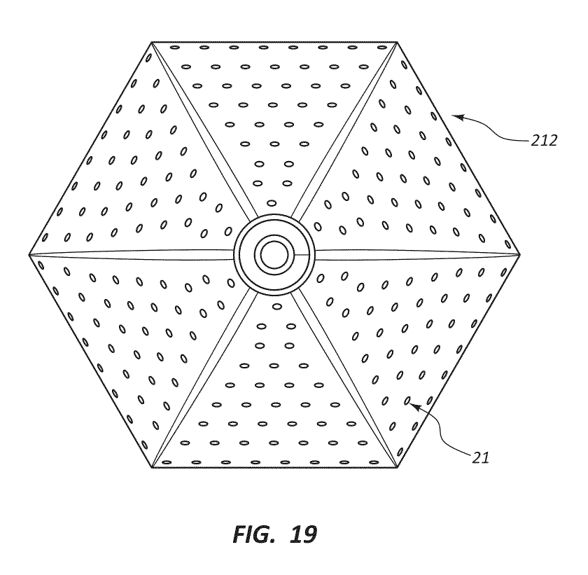

FIG. 19 shows a three-dimensional top view elevation of a coned floor component 212, which guides organic materials towards a center, vertical auger 220. Also shown in this embodiment of the invention are hot recycle gas pressure injection valves 21.

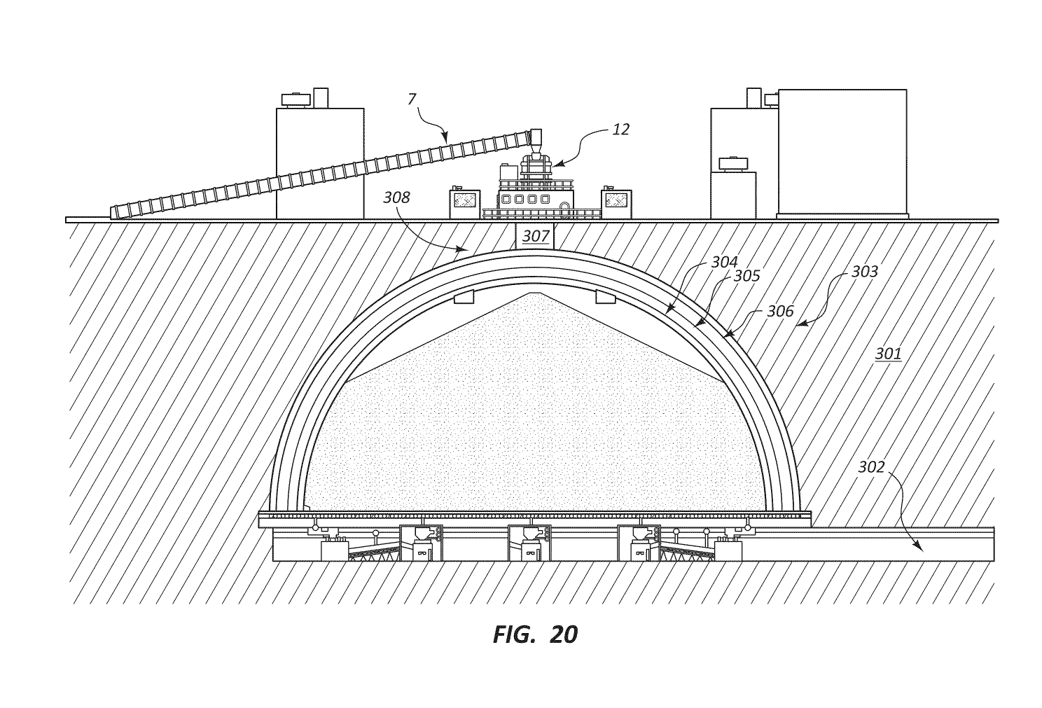

FIG. 20 is a side elevation cut away of a dome buried within a formation in this embodiment. Layers of aggregate and clay are depicted as well us all underground tunnel whereby processed organic material may be exited from beneath the dome retort.

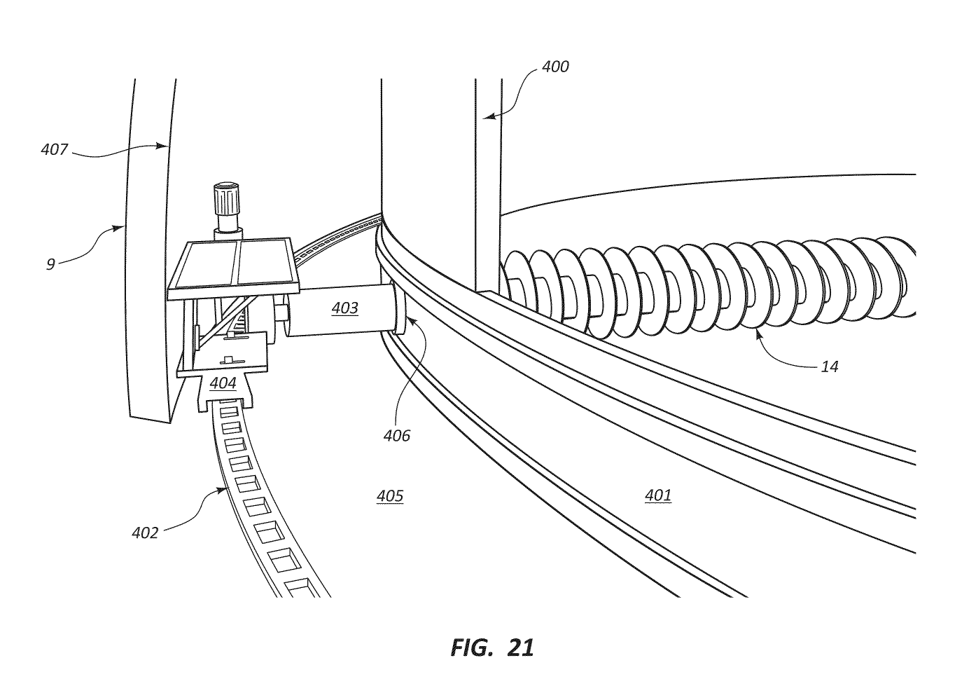

FIG. 21 is a side elevation of a dome retort within a formation 301 wherein in this embodiment, a dome retort with reinforced, high temperature concrete inner shell 304 is covered by aggregate layer 305 and clay layer 306 for thermal and permeability control. Feed chute 307 provides penetration through formation 301 to reach the dome retort. Vapor recovery pipes 308 provide access to remove vapor hydrocarbons being produced. Sealed conveyor 7 is at surface and feeds a surface based, sealed vapor lock hopper 12. Following the processing of the organic material within the dome retort, exit tunnel 302 provides a tunnel for conveyors to remove organic materials.

Whereby horizontal floor auger 14, extends through a rotary bearing 406 mounted in vapor sealing connection to a sliding barrier wall 401, which encircles the perimeter of the dome retort 9, and rotates circularly beneath interim retort wall 400. Sliding barrier wall is sealed, eliminating any vapor escape between dome retort heating zone and a perimeter tunnel 405. Perimeter tunnel has horizontal auger track 402 embedded in its concrete floor which is mechanically oriented to a auger rotary gear drive system 404 to advance forward the horizontal floor auger 14 and 403. As floor auger 14 and 403 rotates and advances, sliding bearing wall 401, which encircles the perimeter of the dome retort, advances as well, rotating circularly in the sealed bearing 406. Perimeter tunnel 407 is maintained at positive pressure of inert gases of either nitrogen or carbon dioxide to ensure no spark from rotary gear crank 402 slipping, or from possible spark of rotary gear shaft motor drive system 404. Rotary gear shaft track 402 may also be submerged in a high temperature fluid so as to suppress any spark created from accidental slippage of the rotary gear drive system 404.

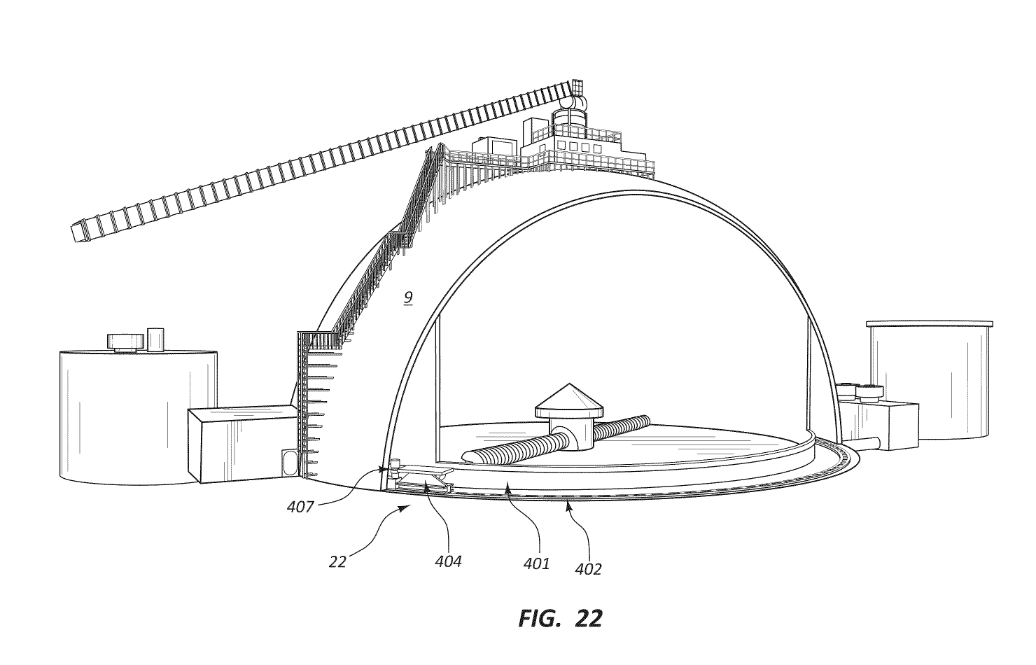

FIG. 22 is a side elevation of a dome retort 9 wherein in this embodiment, a perimeter tunnel room provides a clean room for a perimeter auger track and its motor drive unit 407. Dome retort floor 22, supports a sliding barrier wall 401, which encircles the perimeter of the dome retort and rotates circularly. Perimeter tunnel has horizontal auger track 402 embedded in its concrete floor which is mechanically oriented to a auger rotary gear drive system 404 to advance forward the horizontal floor auger.

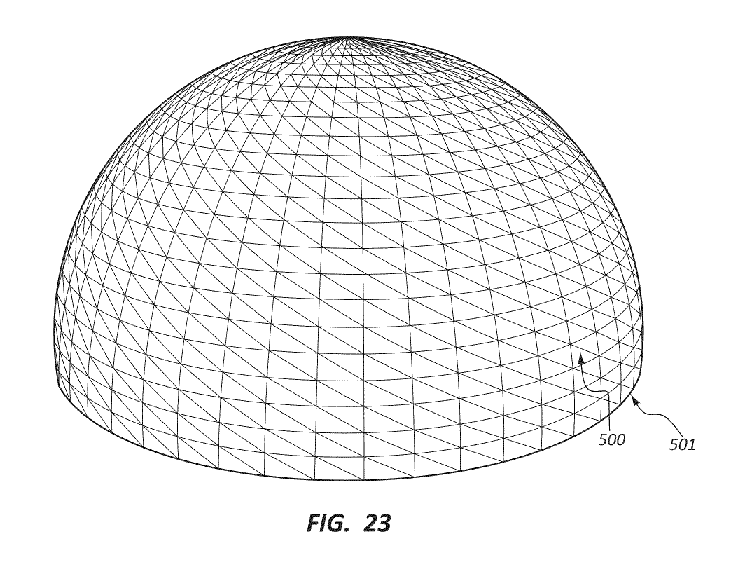

FIG. 23 is an internal geodesic frame 501 bolted to the internal side of the dome retort (not shown). Geometric panels 500 of any shape may be constructed and bolted to internally supported geodesic frame 501. It should be understood that within this embodiment or others, any geodesic frame of any shape or configuration may provide internal support to panels which provide vapor sealing within any dome retort of the invention.

Residence time of organic material within a hydrocarbon extraction system of an embodiment of the present invention is contemplated to comprise a time period of between a few minutes up to over 100 days, and retorting of the organic material is contemplated to be conducted at a temperature of from about 700° F. to about 1200° F. and, more specifically, between about 750° F. and 950° F.

It is contemplated that the process isolation barrier within or constructed as the monolithic dome may thermally isolate a chamber in which the hydrocarbon extraction process is conducted sufficiently to reduce the temperature of an external surface of the monolithic dome to about 400° F. or less, or even 200° F. or less.

Prior to exiting the dome retort chamber, to avoid vaporization of water in aquifers, other ground water, and any volatiles in the formation surrounding the process barrier, the ore is quenched within water creating steam. The steam can be recycled for reuse in the quenching system after circulating through heat transfer pipes embedded in the floor of the dome retort delivering heat energy to the floor of the dome floor mass via conduction.

The dome structure may be constructed by, for example, first inflating an airform having a shape corresponding to the desired shape of the dome structure to be formed. Polyurethane or another polymer material then may be sprayed onto the inner surface of the inflated airform and allowed to solidify, thereby forming a relative stable dome-shaped structure. Steel rebar or other reinforcing material then may be applied to the inner surface of the polymer material, after which shotcrete or other cement-like refractory material may be applied to the inner surface of the dome-shaped structure and over the steel rebar or other reinforcing material. A dome structure may also be fabricated without use of an airform. Construction without an airform may include the stacking of sand into a dome shape, construction of the dome over the dome-shaped surface of the sand structure, and subsequent removal of sand from within the dome following dome construction. Alternatively, a dome may be constructed as a geodesic dome using welded or otherwise sealed geodesic patterns or geometries which create enclosures substantially in the shape of a dome, ellipse or crenosphere.

In some embodiments, the dome structure may comprise a dome structure fabricated as disclosed in any of U.S. Pat. No. 4,155,967, which issued May 22, 1979 to South et al., U.S. Pat. No. 4,324,074, which issued Apr. 13, 1982 to South et al., U.S. Pat. No. 5,918,438, which issued Jul. 6, 1999 to South, U.S. Pat. No. 6,203,261, which issued Mar. 20, 2001 to South et al., and U.S. Pat. No. 7,013,607, which issued Mar. 21, 2006 to South, the disclosures of which patents are incorporated herein in their entireties by this reference.

In one embodiment, a dome retort may be constructed in tandem with a deep basement or excavation creating a sloped lower portion. Combined, the dome retort and a basement may substantially increase the interim volume of the dome retort area. Beneath such excavations can still exist yet further basements and tunnels containing conveyors, piping, control, maintenance and auger and discharge and quenching equipment.

The liner for the process isolation barrier constructed as a dome retort may comprise one or more linings of a dome or interior geodesic liner fabricated of steel, corrugated pipes, pipes, conduits, rolled steel, clay, bentonite clay, compacted fill, volcanic materials, refractory cement, cement, synthetic geogrids, fiberglass, rebar, tension cables, nano-carbons, high temperature cement, gabions, meshes, rock bolts, steel anchors, rebar, shot-crete, filled geotextile bags, plastics, cast concrete pieces, wire, cables, polymers, polymer forms, styrene forms, bricks, insulation, ceramic wool, drains, gravel, tar, salt, sealants, pre-cast panels, pre-cast concrete, in-situ concrete, polystyrene forms, steel mats, abrasion resistant materials, tungsten carbide, or combinations thereof.

The liner of the process isolation barrier which is the dome retort may be fabricated using pre-cast concrete sections or pre-welded mesh sections, assembled as a geodesic dome to form a barrier within a dome or as a dome itself. Such sections may be constructed with or without an air-form balloon.

The liner of the process isolation barrier constructed as a dome may be fabricated to act as a barrier to ground water within an adjacent geological formation. The liner of the process control barrier of the dome retort may be constructed or placed in direct contact with a wall of an excavation or formation to comprise a barrier between an interim of the process isolation barrier and the face of an adjacent formation.

The top cap of the process isolation barrier of the dome itself spans the domes floor and is structurally self-supporting, exerting pressure to an encircling stem wall, footing, surface, floor or basement. The dome may be constructed of concrete, steel, cement, reinforcement, hooped reinforcement, mesh, clay, sand, gravel, tension cables, rebar, beams, polyurethane foams, insulations, inflated forms, geodesic steel configurations, or combinations thereof. The top of the dome may include a hole for further sealing with charge feeder, lock hopper or vapor sealed lock hopper equipment.

Feedstock material may be provided by excavating organic material from a deposit adjacent to the dome retort. Alternatively, the organic material may be sourced from a location remote from the location of the dome retort. The organic material so extracted may be comminuted prior to introduction into the dome retort for processing. The organic material may be sized to an approximate particle size of between ¼ inch and 36 inches. The organic material collectively may exhibit a void space of from about 10% to about 50% of a total volume thereof during descent thereof through the process isolation barrier.

To better illustrate the scope of the invention, the organic material may be selected to comprise oil shale, coal, lignite, tar sands, peat, bio mass, wood chips, algae, corn stover, castor plants, sugar cane, hemp plants, used tires, bast fiber family plants, oil sands, tar sands, waste materials, garbage, animal waste, or a combination thereof.

The organic material to be processed may be introduced into the at least one dome retort to descend therein substantially by gravity. For example by use of a vapor sealing lock hopper. The vapor sealing lock hopper may be mounted to the top of the dome retort process isolation barrier to introduce the organic material therethrough.

Heat energy for hydrocarbon extraction may be provided by combustion of the organic materials, combustion of hydrocarbons, combustion of hydrocarbons removed from the organic material, burners, a solid oxide fuel cell, a fuel cell, waste heat from an adjacent facility, a solar based heat transfer fluid, an electrical resistive heating, solar sources, nuclear power, geothermal, oceanic wave energy, wind energy, a microwave heat source, steam, a super heated fluid, or any combination thereof. If hydrocarbons removed from the organic material are combusted, at least one of sulfur and nitrogen may be removed therefrom prior to combustion. Heat for hydrocarbon extraction may be substantially continuously applied, in keeping with the continuous nature of the extraction process, and varied as desired to enhance process conditions.

The application of heat may include injecting heated gases into the at least one dome retort through which the organic material passes such that the organic material passing through the at least one dome retort is heated via convection as the organic material descends and heated gases are allowed to pass throughout the dome retort. The injected heating gases may be recycled gases recovered from the hydrocarbon extraction, and the recycled gases may be reheated prior to injection into the dome retort.

To enhance processing, the organic material may be heated with elements of a heated, solid material that is separate from the organic material. The elements of heated, solid material may comprise heated sand, heated ceramic balls, hollowed ceramic balls, marbles, organic material containments, heated rocks, heat steel balls, or combinations thereof. The elements of solid material, after heat transfer to the organic material, may be recovered for reheating.

The application of heat may also be effected by transferring heat from a heat transfer fluid through a wall or floor of the dome retort and its process isolation barrier, such as from a conduit within or atop its floor.

The application of heat may comprise heating the organic material sufficiently within a temperature range to substantially avoid formation of carbon dioxide or non-hydrocarbon leachates.

After hydrocarbon extraction therefrom, removal of organic material from the dome retort may be effected through a vapor sealed lock hopper passing ore down to a sealed quenching or cooling chamber. Heat from the organic material may be recovered for reuse in the extraction process, including delivery through heat transfer pipes in the floor or otherwise.

Heat may be removed from the organic material by introducing heated organic material after the hydrocarbon extraction into a separate cooling chamber vertically positioned below heated elevations (dome retort) to remove heat from the organic material via means of a heat transfer method. The heat transfer method may comprise the generation of steam, rinsing, air, blowers, heat exchangers, heat transfer fluids, heat transfer conduits, gases, heat transfer conduits with fluidly connected heat transfer fluids, the introduction of solids, heat exchangers, solids to absorb heat, or any combination thereof. Steam generated in the heat transfer method may be used to generate electricity. The transfer of heat, if effected via heat transfer fluids within a conduit connected to the cooling chamber may employ a conduit extending to another chamber within the dome retort or to a preheating conveyor or an adjacent retorting or preheating dome.

Further, a heat transfer fluid may be circulated throughout a portion of the dome beneath a primary heating area such as a preheat dome or a retort dome to at least partially recover heat or hydrocarbons from the organic material.

For some applications, heat within a given dome may be transferred to another dome. Such transfer may be used, for example, to facilitate startup of a hydrocarbon extraction system within the second dome.

The organic material removal of organic material following the extraction of hydrocarbons therefrom may be accomplished via conveyance through a tunnel proximate and connected to the dome retort proximate the lower end thereof. By way of non-limiting example, the tunnel may be constructed of arched corrugated panels with rebar and concrete for reinforcement. The tunnels may have vapor sealing exits allowing the tunnels to be flooded with inert gases such as nitrogen and carbon dioxide. Further, the tunnels and all chambers in association within or below the dome retort may be pressurized such that hydrocarbons escaping from the dome retort cannot enter these areas where electrical equipment, motors and possible ignition sources reside. The tunnels beneath dome may be exited to a location which is a hillside, embankment, cliff, outcrop, ledge or escarpment.

It may be desirable to prevent agglomeration of the organic material at least during the hydrocarbon extraction. By way of non-limiting example, agglomeration may be prevented using chutes, cables, fins, channels, admixes, sizing, mixtures, flutes, beams, riffles, baffles, spirals, ceramic balls, alloy balls, marbles, casings, sonic cavitations, vibratory plates, gases, pressurized gases, vibratory walls, vibration, steel constructions, sand, chimneys, segregation, partitions, screens, meshes, posts, separate chambers, augers, reclaimers, floor reclaimers or any combination thereof. Means to prevent agglomeration as modular units may be disposed or assembled within the shaft.

At least part of the process of hydrocarbon extraction may be performed at above atmospheric pressure. Similarly, at least part of the process of hydrocarbon extraction may be performed below atmospheric pressure.

At least a portion of the retorting vessel interior may be treated with an anti-abrasion protective means. At least a portion of the anti-abrasion means may comprise tungsten carbide.

The process isolation barrier in which the hydrocarbon extraction process is conducted may comprise segregated chambers within the dome retort itself. The segregated chambers may be comprised of preheating chambers, flashing chambers, retorting chambers, combustion chambers, soaking chambers, rinsing chambers, steam chambers, collection chambers, stirring chambers, drying chambers, cooling chambers, heat transfer chambers, loading chambers or any combination thereof.

Conduits for control, heat transfer, extracted hydrocarbon transport, drainage or other purposes may be placed or formed within the domes lining infrastructure, floor or basement walls or through lateral and perimeter tunnels of the process isolation barrier of the dome retort.

Collection of hydrocarbons removed from the organic material includes cooling the collected hydrocarbons, such as with a condenser. The condenser may be used to separate non-condensable hydrocarbons subsequently used to create heat for the at least one retorting or preheating dome.

Collecting the extracted hydrocarbons may include the extraction of gases at or near the top of a dome retort, the extraction of liquids at two or more elevations within the dome retort, or both. The extraction of hydrocarbon liquids at two or more elevations within the process isolation shaft may be employed to mutually segregate at least two of hydrogen, propane, butane, methane, naptha, diesel, distillate, kerosene, residual, or gas oil fractions. This may be accomplished by constructing an internal vacuum tower penetrating the piled permeable body of the organic material with the dome retort.

Collecting the extracted hydrocarbons may comprise the use of at least one conduit embedded within a wall or floor of the dome retort. Conduits may be used separately for heat transfer as well as for recycle gas injection. Other conduits embedded or penetrating the floor of the dome retort may be used to create a fluidized bed floor such that the floor is sloped between 6 and 10 degrees. Injected hot gases (instead of air) would be the preferred fluidizing vapor. Such vapor may have capacity to push sludge and particles to slurry pumps at drain locations.

The introduction of or removal of the organic material into the process isolation barrier is accomplished by conveying the organic materials into a vapor sealed lock hopper atop or below the dome retort. ill this manner the dome retort hot gases and temperature remain within the dome yet the organic material is charged and exited following hydrocarbon extraction from the organic materials.

During heating and extraction a hydrogen donor agent may be injected into the dome to hydrogenate the hydrocarbons, To facilitate this process, a catalyst may be dispersed or mixed with the organic material in sufficient quantities such that the hydrogenation or partial hydrotreating of the hydrocarbons may occur within the dome retort. The hydrogen donor agent may be natural gas or hydrogen, methane or be comprised of other distilled hydrocarbons from an atmospheric tower or vacuum tower bottoms or bitumen and conditions of pressure and temperature are controlled or provided sufficient to cause reforming of the hydrocarbons to produce an upgraded hydrocarbon product.

Following collection of the extracted hydrocarbons from the dome retort, they may be placed in a tank or an oil containing dome, vessel or tank, to form a body of liquid hydrocarbons. Such containments may have further hydrogen donor agent circulated into the body of liquid hydrocarbons to further upgrade the liquid hydrocarbons.

Collecting the extracted hydrocarbons may include collecting a liquid product from a lower region of the dome retort and collecting a gaseous product from an upper region of the dome retort structure. Collecting a gaseous product from the dome retort structure may further comprise directing the gaseous products to be heated and recycled through the dome retort at or near the floor level or injected through conduits within the dome or from its walls at different elevations. Such gases may be recycled gas recycling multiple times through the dome retort. The recycle gases may be heated to a temperature between 700 degrees Fahrenheit and 1,200 degrees Fahrenheit and, in one embodiment, are injected at the floor level through pipes embedded in the floor of the dome retort.

When heat is provided into the dome retort structure, it is envisioned that the creating heat energy will utilize means to reduce emissions of carbon monoxide, particle matter, carbon dioxide, nitrous oxide, sulfur dioxins, or combinations thereof. The providing of heat energy by hydrocarbon combustion may also be conducted under stoichiometric conditions of fuel to oxygen for other emission benefits. Emission reduction may also comprise sequestering carbon dioxide created as a result of application of heat to the organic material by geological sequestration, oceanic sequestration, sequestration into brine liquid, enhanced oil recovery well injection, or combinations thereof. In one embodiment of carbon dioxide sequestration, a cement additive from the sequestered carbon dioxide is created in brine liquid. Following drying of such additive, the additive may be used with spent tailings or in the concrete construction of additional dome retorts.

The dome retort structure may be used to produce liquids containing one or more of kerogen from oil shale, coal liquids, biomass liquids, oil sands liquids, liquids from lignite, liquids from animal waste, liquids from waste materials, liquids from tires, or combinations thereof. In one embodiment, the dome retorts are situated adjacent to refineries and upgraders which share hydrotreating, hydrocracking, distillation and vacuum distillation process equipment. It is envisioned that the recycling of various liquid components or solid components from such process equipment may be reintroduced into the dome retort for further pyrolysis.

Following pyrolysis, the removal of organic material subsequent to hydrocarbon extraction is effected after cooling or the organic material through a quenching process which may be within a sealed auger system. Once the organic material is lowered to a more reasonable temperature, it may be conveyed by normal conveyance systems and placed within a tailings management impoundment. Such an impoundment may comprise an encapsulated infrastructure constructed of one or more of steel, corrugated pipes, pipes, conduits, rolled steel, clay, bentonite clay, compacted fill, volcanic materials, refractory cement, cement, synthetic geogrids, fiberglass, rebar, nano-carbon reinforced cement, glass fiber filled cement, high temperature cement, gabions, meshes, rock bolts, rebar, shot-crete, filled geotextile bags, plastics, cast concrete pieces, wire, cables, polymers, polymer forms, styrene forms, bricks, insulation, ceramic wool, drains, gravel, sand, tar, salt, sealants, pre-cast panels, liners, pumps, drains or combinations thereof. The encapsulated infrastructure provides a long term sequestration of organic material from fresh water hydrology, rivers, streams, wildlife, drainages, lakes, plants or combinations thereof.

In one embodiment of the invention, leaching a solvent through the organic material subsequent to hydrocarbon extraction can be performed. The solvent can be a solvent for the extraction of one or more target materials comprising precious metals, noble metals, iron, gold, copper, uranium, aluminum, platinum, nickel, palladium, molybdenum, cobalt, sodium bicarbonate, nacholite, or combinations thereof. Given the acidic and corrosive nature of leachates or solvents, this step or process may be carried out in an adjacent dome specifically lined or constructed to withstand such corrosion or within the dome retort itself. In this regard not only are hydrocarbons recovered but other precious target materials as well.

When the organic material introduced into the dome retort is crushed oil shale and the application of heat is conducted under time and temperature conditions is sufficient, liquid hydrocarbon product having an API from about 27 to about 45 may be produced.

When the organic material introduced into the dome retort is coal and the application of heat is conducted under time and temperature conditions sufficient to form a liquid hydrocarbon, a product having an API gravity from about 16 to about 35 may be produced.

In one embodiment, the size of the dome retort allows for high volume retorting yet provides longer heating residence time of the organic material over previously known methods. In some embodiments, heating times may be from about 5 minutes to about 95 days prior to removing the organic material from the dome retort, which is much longer than other retorts, yet the volume is far greater than other known retorts. The application of heat to the feedstock material within the dome retort structure may be controlled using a computer control system comprising a processor, memory, and a computer program stored in the memory that is configured to maintain a substantially continuous temperature of between ambient temperature and about 1200 F within one or more regions within the dome retort structure.

Auger systems including floor reclaimers and augers also may be controlled using the control system, as may be the conveyors and vapor sealed lock hopper feeding the dome retort. The volume rate of organic material can be precisely controlled.

In one embodiment, a screen or magnetic separate is used to disallow any particle larger than a permitted size or any metallic particle from entering the dome retort.

For safety, the dome retort structure may include a system for purging the dome retort extraction environment with an inert gas which may include one or more of carbon dioxide and nitrogen gas. Similarly, these inert gases may be used throughout affiliated rooms, basement, tunnels, storage, access, mechanical and channels comprising mechanical, electrical and controls for the dome retort. A positive pressure may be maintained in these areas so as to prevent the escape or communication of such area with hydrocarbon vapors from the dome retort structure. Such purging may remove oxygen which, in combination with hydrocarbons, may result in an explosion or uncontrolled combustion.

The dome retort is comprised of a shell or isolation barrier formed of steel, corrugated pipes, pipes, conduits, rolled steel, clay, bentonite clay, compacted fill, volcanic materials, refractory cement, cement, synthetic geogrids, fiberglass, glass fibers, rebar, tension cables, nano-carbons, high temperature cement, gabions, meshes, rock bolts, shot-crete, filled geotextile bags, plastics, cast concrete pieces, wire, cables, polymers, polymer forms, styrene forms, bricks, insulation, ceramic wool, drains, gravel, tar, salt, sealants, pre-cast panels, liners, abrasion resistant materials, tungsten carbide, or combinations thereof. The dome shell may be constructed as a substantially monolithic shell and may be comprised of multiple shells within one another. The shells may be then buried beneath the ground in layers of sand, aggregate, clay and liners made of any material for further permeability control. Because the dome retort is fixed and organic materials are passing through, its isolation barrier is a reusable structure for passing organic material into and out of the at least one dome retort.

In one embodiment, at least one retort dome structure may contain a plurality of conduits disposed within the permeable body of the organic material such that the conduits are being configured as heating pipes. Similarly, in at least one cooling dome retort at least a portion of the plurality of conduits is oriented within the permeable body of the organic material so as to remove heat prior to quenching. At least a portion of the conduits is envisioned to be positioned vertically so as to allow the organic material to flow and reduce static pressure from the organic material on the conduits.

The one or more present inventions, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure.

The present invention, in various embodiments, includes providing devices and processes in the absence of items not depicted and/or described herein or in various embodiments hereof, including in the absence of such items as may have been used in previous devices or processes, e.g., for improving performance, achieving ease and/or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method of obtaining hydrocarbons from organic material, comprising:
introducing organic material into an at least substantially air tight dome retort structure comprising an at least one substantially monolithic dome through a vapor sealing delivery structure;
heating the organic material to an elevated temperature and causing hydrocarbons to be released from the organic material;
removing the organic material from the at least substantially air tight dome retort structure using at least one mechanical auger; and
collecting hydrocarbons released from the organic materials.

2. The method of claim 1, wherein the method of heat is delivered by heated injection gases delivered through a conduit.

3. The method of claim 2, wherein the injection gas delivering heat is a recycled gas recovered from near the upper portion of the substantially air tight dome retort.

4. The method of claim 3, wherein the recycled gas has been reheated prior to reinjection into the substantially air tight retort.

5. The method of claim 1, wherein the method of heat injection also comprises the use of a pressure actuated valve.

6. The method of claim 1, wherein the heat is delivered through a conduit intersecting at least a portion of the dome retort floor.

7. The method of claim 1, wherein the floor contains embedded conduits containing heated fluids.

8. The method of claim 6, wherein the mass of the floor radiates heat upwardly.

9. The method of claim 1, wherein the floor contains embedded conduits which inject upwardly through a pressure valve heated fluids.

10. The method of claim 1, wherein the floor is sloped sufficient for the purpose of collecting hydrocarbons by gravity.

11. The method of claim 1, wherein the organic material is comprised of oil shale, coal, lignite, waste material, animal waste, biomass, tar sands, oil sands, or combinations thereof.

12. The method of claim 1, wherein the mechanical auger device comprises an auger which rotates as horizontal position above the floor of the retort.

13. The method of claim 12, wherein the mechanical auger device intersects a substantially vapor sealed perimeter chamber.

14. The method of claim 13, wherein the substantially vapor sealed perimeter chamber contains floor track for the auger.

15. The method of claim 13, wherein the substantially vapor sealed perimeter chamber is maintained at an atmospheric pressure greater than the dome retort.

16. The method of claim 1, wherein the organic material is mechanically augured through at least one hole in the floor passing through a fluid seal control means.

17. The method of claim 16, wherein the fluid sealing means comprises a vapor sealing lock hopper.

18. The method of claim 16, wherein the fluid sealing means comprises a quenching chamber.

19-116. (canceled)

117. A system for extracting hydrocarbons from organic material, the system comprising:
a substantially monolithic dome capping a subterranean chamber;
a process isolation barrier liner surrounding the subterranean chamber below the substantially monolithic dome;
at least one layer of aggregate material overlying the dome;
at least one apparatus configured for introducing organic material into the subterranean chamber through an upper end of the dome, the at least one apparatus configured for preventing escape of vapor from the subterranean chamber through the dome;
at least one retort vessel located within the subterranean chamber and configured to receive the organic material therein; and
a control system comprising a computer program stored in memory of the control system, the computer program configured to control at least one operating process parameter within the at least one retort vessel.

118. A system for extracting hydrocarbons from organic material, the system comprising:
a dome structure defining a cap over a subterranean chamber;
an isolation barrier below the cap and surrounding the subterranean chamber, the isolation barrier in contact with a surface of at least one earth formation, a periphery of the dome structure in substantially sealed engagement with the isolation barrier;
at least one apparatus for introducing organic material into an upper end of the subterranean chamber through the dome and configured for preventing substantial escape of vapor from the subterranean chamber through the dome;
at least one preheat vessel located within the dome and configured to receive the organic material therein;
at least one retort vessel located within the dome and configured to receive the organic material therein from the at least one preheat vessel;
at least one fabricated cooling chamber configured to receive the organic material from the at least one retort vessel;
at least one quenching chamber configured to receive the organic material from the at least one cooling chamber;
at least one device for collecting hydrocarbons extracted from the organic material; and
a control system comprising a computer program stored in memory of the control system, the computer program configured to control at least one retorting process operating parameter within the subterranean chamber.
